SEQUENCE LISTING (21 sequences)

SEQ ID NO: 1 - DNA, 1041 bp, Escherichia coli
  ompA nucleic acid sequence, 1041 bp
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacctggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaa

SEQ ID NO: 2 - protein, 346 aa, Escherichia coli
  OmpA preprotein amino acid sequence, 346 aa
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 3 - protein, 325 aa, Escherichia coli
  OmpA mature protein amino acid sequence, 325 aa
APKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 4 - DNA, 237 bp, Escherichia coli
  lpp nucleic acid sequence, 237 bp
atgaaagctactaaactggtactgggcgcggtaatcctgggttctactctgctggcaggttgctccagcaacgctaaaatcgatcagctgtcttctgacgttcagactctgaacgctaaagttgaccagctgagcaacgacgtgaacgcaatgcgttccgacgttcaggctgctaaagatgacgcagctcgtgctaaccagcgtctggacaacatggctactaaataccgcaagtaa

SEQ ID NO: 5 - protein, 78 aa, Escherichia coli
  Lpp preprotein amino acid sequence, 78 aa
MKATKLVLGAVILGSTLLAGCSSNAKIDQLSSDVQTLNAKVDQLSNDVNAMRSDVQAAKDDAARANQRLDNMATKYRK

SEQ ID NO: 6 - protein, 58 aa, Escherichia coli
  Lpp mature protein amino acid sequence, 58 aa
CSSNAKIDQLSSDVQTLNAKVDQLSNDVNAMRSDVQAAKDDAARANQRLDNMATKYRK

SEQ ID NO: 7 - DNA, 522 bp, Escherichia coli
  pal nucleic acid sequence, 522 bp
atgcaactgaacaaagtgctgaaagggctgatgattgctctgcctgttatggcaattgcggcatgttcttccaacaagaacgccagcaatgacggcagcgaaggcatgctgggtgccggcactggtatggatgcgaacggcggcaacggcaacatgtcttccgaagagcaggctcgtctgcaaatgcaacagctgcagcagaacaacatcgtttacttcgatctggacaagtacgatatccgttctgacttcgctcaaatgctggatgcacatgcaaacttcctgcgtagcaacccgtcttacaaagtcaccgtagaaggtcacgcggacgaacgtggtactccggaatacaacatctccctgggtgaacgtcgtgcgaacgccgttaagatgtacctgcagggtaaaggcgtttctgcagaccagatctccatcgtttcttacggtaaagaaaaacctgcagtactgggtcatgacgaagcggcatactccaaaaaccgtcgtgcggtactggtttactaa

SEQ ID NO: 8 - protein, 173 aa, Escherichia coli
  Pal preprotein amino acid sequence, 173 aa
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 9 - protein, 152 aa, Escherichia coli
  Pal mature protein amino acid sequence, 152 aa
CSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 10 - DNA, 921 bp, Escherichia coli
  ybiS nucleic acid sequence, 921 bp
atgaatatgaaattgaaaacattattcgcagcggccttcgctgttgtcggcttttgcagtaccgcctctgcggtaacttatcctctgccaaccgacgggagtcgcctggttggtcagaatcaggtgatcaccattcctgaaggtaacactcagccgctggagtattttgccgccgagtaccagatggggctttccaatatgatggaagcgaacccgggtgtggataccttcctgccgaaaggcggtactgtactgaacattccgcagcagctgatcctgccggataccgttcatgaaggcatcgtcattaacagtgctgagatgcgtctttattactatccgaaagggaccaacaccgttatcgtgctgccgatcggcattggtcagttaggcaaagatacgcctatcaactggaccaccaaagttgagcgtaaaaaagcaggcccgacctggacgccgaccgccaaaatgcacgcagagtaccgcgctgcgggcgaaccgcttccggctgtcgttccggcaggtccggataacccgatggggctgtatgcactctatatcggtcgcctgtatgctatccatggcaccaacgccaacttcggtatcggcctgcgtgtaagtcatggttgtgtgcgtctgcgtaacgaagacatcaaattcctgttcgagaaagtaccggtcggtacccgcgtacagtttattgatgagccggtaaaagcgaccaccgagccagacggcagccgttatattgaagtccataacccgctgtctaccaccgaagcccagtttgaaggtcaggaaattgtgccaattaccctgacgaagagcgtgcagacagtgaccggtcagccagatgttgaccaggttgttcttgatgaagcgattaaaaaccgctccgggatgccggttcgtctgaattaa

SEQ ID NO: 11 - protein, 306 aa, Escherichia coli
  YbiS protein amino acid sequence, 306 aa
MNMKLKTLFAAAFAVVGFCSTASAVTYPLPTDGSRLVGQNQVITIPEGNTQPLEYFAAEYQMGLSNMMEANPGVDTFLPKGGTVLNIPQQLILPDTVHEGIVINSAEMRLYYYPKGTNTVIVLPIGIGQLGKDTPINWTTKVERKKAGPTWTPTAKMHAEYRAAGEPLPAVVPAGPDNPMGLYALYIGRLYAIHGTNANFGIGLRVSHGCVRLRNEDIKFLFEKVPVGTRVQFIDEPVKATTEPDGSRYIEVHNPLSTTEAQFEGQEIVPITLTKSVQTVTGQPDVDQVVLDEAIKNRSGMPVRLN

SEQ ID NO: 12 - DNA, 963 bp, Escherichia coli
  ycfS nucleic acid sequence, 963 bp
gtgatgatcaaaacgcgtttttctcgctggctaacgttttttacgttcgccgctgccgtggcgctggcgctaccggcaaaagccaacacctggccgctgccgccagcgggcagtcgtctggttggcgaaaacaaatttcatgtggtggaaaatgacggtggttctctggaagccatcgccaaaaaatacaacgtcggctttctcgctctgttacaggctaaccccggcgttgatccttacgtaccgcgcgcgggcagcgtgttaacgatcccgttgcaaaccctacttccagatgcgccgcgcgaaggcattgtgatcaacattgcggagctgcgtctctattactacccgccgggtaaaaattcggtaaccgtgtatccaataggtattggtcagttaggtggtgacacgctgacaccgacaatggtgaccaccgtttcagacaaacgtgcaaacccaacctggacgccaacggcaaacatccgcgcccgttataaagcacagggaattgagttgcctgcggtagtgccggctggactggataacccaatgggccatcatgcgattcgtctggcggcctatggcggcgtttatttgcttcatggtacgaacgccgatttcggcattggcatgcgggtaagttctggctgtattcgtctgcgggatgacgatatcaaaacactctttagccaggtcaccccaggcaccaaagtgaatatcatcaacactccgataaaagtctctgccgaaccaaacggtgcgcgtctggttgaagtacatcagccgctgtcagagaagattgatgacgatccgcagctgctgccaattacgctgaatagcgcaatgcaatcatttaaagatgcagcacaaactgacgctgaagtgatgcaacatgtgatggatgtccgttccgggatgccggtggatgtccgccgtcatcaagtgagcccacaaacgctgtaa

SEQ ID NO: 13 - protein, 320 aa, Escherichia coli
  YcfS protein amino acid sequence, 320 aa
VMIKTRFSRWLTFFTFAAAVALALPAKANTWPLPPAGSRLVGENKFHVVENDGGSLEAIAKKYNVGFLALLQANPGVDPYVPRAGSVLTIPLQTLLPDAPREGIVINIAELRLYYYPPGKNSVTVYPIGIGQLGGDTLTPTMVTTVSDKRANPTWTPTANIRARYKAQGIELPAVVPAGLDNPMGHHAIRLAAYGGVYLLHGTNADFGIGMRVSSGCIRLRDDDIKTLFSQVTPGTKVNIINTPIKVSAEPNGARLVEVHQPLSEKIDDDPQLLPITLNSAMQSFKDAAQTDAEVMQHVMDVRSGMPVDVRRHQVSPQTL

SEQ ID NO: 14 - DNA, 933 bp, Escherichia coli
  erfK nucleic acid sequence, 933 bp
atgcgtcgtgtaaatattctttgctcatttgctctgctttttgccagccatactagcctggcggtaacttatccattacctccagagggtagccgtttagtggggcagtcgtttactgtaactgttcctgatcacaatacccagccgctggagacttttgccgcacaatacgggcaagggttaagtaacatgctggaagcgaacccgggcgctgatgtttttttgccgaagtctggctcgcaactcaccattccgcagcaactgattttgcccgacactgttcgtaaagggattgttgttaacgtcgctgagatgcgtctttattactacccaccagacagtaatactgtggaagtctttcctattggtatcggccaggctgggcgagaaaccccgcgtaactgggtgactaccgttgaacgtaaacaagaagctccaacctggacgccaacgccgaacactcggcgcgaatatgcgaaacgaggggagagtttgcccgcatttgttcctgcgggccccgataatcccatggggctgtacgcgatttatattggcaggttgtatgccatccatggtaccaatgccaattttggtattgggctccgggtaagtcagggctgtattcgtctgcgcaatgacgatatcaaatatctgtttgataatgttcctgttgggacgcgtgtgcaaattatcgaccagccagtaaaatacaccactgaaccagatggctcgaactggctggaagttcatgagccattgtcgcgcaatcgtgcagaatatgagtctgaccgaaaagtgccattgccggtaaccccatctttgcgggcgtttatcaacgggcaagaagttgatgtaaatcgcgcaaatgctgcgttgcaacgtcgatcgggaatgcctgtgcaaattagttctggttcaagacagatgttttaa

SEQ ID NO: 15 - protein, 310 aa, Escherichia coli
  ErfK protein amino acid sequence, 310 aa
MRRVNILCSFALLFASHTSLAVTYPLPPEGSRLVGQSFTVTVPDHNTQPLETFAAQYGQGLSNMLEANPGADVFLPKSGSQLTIPQQLILPDTVRKGIVVNVAEMRLYYYPPDSNTVEVFPIGIGQAGRETPRNWVTTVERKQEAPTWTPTPNTRREYAKRGESLPAFVPAGPDNPMGLYAIYIGRLYAIHGTNANFGIGLRVSQGCIRLRNDDIKYLFDNVPVGTRVQIIDQPVKYTTEPDGSNWLEVHEPLSRNRAEYESDRKVPLPVTPSLRAFINGQEVDVNRANAALQRRSGMPVQISSGSRQMF

SEQ ID NO: 16 - DNA, 483 bp, Escherichia coli
  yfiB nucleic acid, 483 bp
atgataaagcacctggtagcacccctggttttcacctcactaatactgactggctgccagtcccctcagggaaagtttactcctgagcaagtcgccgctatgcaatcttatggatttactgaatccgccggcgactggtcgctgggcttatcagatgccattctgttcgcaaaaaatgactacaaattgctcccggaaagccagcaacagatccaaaccatggcagctaaattggcctcgacagggctaacacatgcccgtatggatggacacaccgataactatggtgaagacagttacaacgaaggcttatcattgaaacgggcgaatgtcgtggccgatgcatgggctatgggtggacaaattccacgcagcaatctcaccacacagggtttaggaaaaaaatatcccatagccagtaacaagaccgcccagggccgcgccgagaaccgccgcgtcgcagtggtgattactaccccttaa

SEQ ID NO: 17 - protein, 160 aa, Escherichia coli
  YfiB protein amino acid sequence, 160 aa
MIKHLVAPLVFTSLILTGCQSPQGKFTPEQVAAMQSYGFTESAGDWSLGLSDAILFAKNDYKLLPESQQQIQTMAAKLASTGLTHARMDGHTDNYGEDSYNEGLSLKRANVVADAWAMGGQIPRSNLTTQGLGKKYPIASNKTAQGRAENRRVAVVITTP

SEQ ID NO: 18 - DNA, 660 bp, Escherichia coli
  yiaD nucleic acid, 660 bp
atgaagaaacgtgtttatcttattgccgccgtagtgagtggcgctctggcggtatctggctgcacaactaacccttacaccggcgaacgcgaagcaggtaaatctgctatcggcgcaggtctgggctctctcgtgggcgcgggtattggtgcgctctcttcttcgaagaaagatcgcggtaaaggcgcgctgattggcgcagcagcaggcgcagctctgggcggcggcgttggttattacatggatgtgcaggaagcgaagctgcgcgacaaaatgcgcggcactggtgttagcgtaacccgcagcggggataacattatcctcaatatgccgaacaatgtgaccttcgacagcagcagcgcgaccctgaaaccggcgggcgctaacaccctgaccggcgtggcaatggtactgaaagagtatccgaaaacggcggttaacgtgattggttataccgacagcacgggtggtcacgacctgaacatgcgtctctcccagcaacgtgcggattccgttgccagcgcgttgatcacccagggcgtggacgccagccgcatccgtactcagggccttggcccggctaacccaatcgccagcaacagcaccgcagaaggtaaggcgcaaaaccgccgtgtagaaattaccttaagcccgctgtaa

SEQ ID NO: 19 - protein, 219 aa, Escherichia coli
  YiaD protein amino acid sequence, 219 aa
MKKRVYLIAAVVSGALAVSGCTTNPYTGEREAGKSAIGAGLGSLVGAGIGALSSSKKDRGKGALIGAAAGAALGGGVGYYMDVQEAKLRDKMRGTGVSVTRSGDNIILNMPNNVTFDSSSATLKPAGANTLTGVAMVLKEYPKTAVNVIGYTDSTGGHDLNMRLSQQRADSVASALITQGVDASRIRTQGLGPANPIASNSTAEGKAQNRRVEITLSPL

SEQ ID NO: 20 - DNA, 258 bp, Escherichia coli
  yqhH nucleic acid, 258 bp
atgaaaacgattttcaccgtgggagctgttgttctggcaacctgcttgctcagtggctgcgtcaatgagcaaaaggtcaatcagctggcgagcaatgtgcaaacattaaatgccaaaatcgcccggcttgagcaggatatgaaagcactacgcccacaaatctatgctgccaaatccgaagctaacagagccaatacgcgtcttgatgctcaggactattttgattgcctgcgctgcttgcgtatgtacgcagaatga

SEQ ID NO: 21 - protein, 85 aa, Escherichia coli
  YqhH protein amino acid sequence, 85 aa
MKTIFTVGAVVLATCLLSGCVNEQKVNQLASNVQTLNAKIARLEQDMKALRPQIYAAKSEANRANTRLDAQDYFDCLRCLRMYAE

(19) United States
(12) Patent Application Publication
(10) Pub. No.: US 2023/0340035 A1
(43) Pub. Date: Oct. 26, 2023

(54) GENETICALLY MODIFIED BACTERIUM WITH ALTERED ENVELOP INTEGRITY AND USES THEREOF

(71) Applicant: UNIVERSITÉ CATHOLIQUE DE LOUVAIN, Louvain-la-Neuve (BE)

(72) Inventors: Jean-François COLLET, Hevillers (BE); Michaël DEGHELT, Schaerbeek (BE); Seung Hyun CHO, Bruxelles (BE)

(73) Assignee: UNIVERSITÉ CATHOLIQUE DE LOUVAIN, Louvain-la-Neuve (BE)

(21) Appl. No.: 17/792,011

(22) PCT Filed: Jan. 18, 2021
(86) PCT No.: PCT/EP2021/050969

§ 371 Date: Jul. 11, 2022

(30) Foreign Application Priority Data
Jan. 17, 2020 (EP) 20152513.6

(51) Int. Cl. C07K 14/245; C12P 19/34; C12N 15/70

(52) U.S. Cl. CPC C07K 14/245; C12P 19/34; C12N 15/70

(57) ABSTRACT

Genetically modified gram-negative bacteria, in particular E. coli, that are oversensitive to lysis. These bacteria are therefore useful to improve the yield of nucleic acid extraction, preferably extra-genomic nucleic acid extraction (e.g. plasmid), and/or the yield of a polypeptide, preferably encoded by an extra-genomic nucleic acid. In practice, the strains of E. coli are engineered with a combination of at least 2 mutated genes altering the envelop integrity. More particularly, at least one mutated gene is ompA and at least one mutated gene is a gene involved in Lpp functionality, such as, e.g., the lpp gene, the ybiS gene, the ycfS gene and the erfK gene. These combinations also include mutations in genes that are homologue to the ompA gene, and/or the lpp gene.

### FIELD OF INVENTION

The present invention relates to the field of genetically modified microorganisms, and in particular, genetically modified bacteria having an altered envelop integrity. More precisely, engineered E. coli strains having a combination of mutations in the ompA gene, and the lpp gene or genes encoding polypeptides involved in Lpp function, have been found to be oversensitive to bacterial lysis, as compared to a wild type strain. These bacterial strains are therefore useful in methods for improving nucleic acids and polypeptides production and purification.

### BACKGROUND OF INVENTION

Extra-genomic nucleic acid molecules in general, and plasmids in particular, are genetic elements that are able to replicate in bacteria independently of the bacterial chromosome. Plasmids and plasmid-encoded proteins may advantageously be produced industrially in E. coli strains by fermentation processes, followed by large-scale purification for various applications, including therapeutic applications. Indeed, E. coli has been well characterized since its discovery and numerous tools have been implemented to improve the ease of manipulating this bacterial strain. As a consequence, E. coli strains have been converted into a production plant of choice for the production of nucleic acids and polypeptides.

As an example of therapeutic benefit of plasmids, one may cite immunotherapy and DNA vaccination, for which genetically engineered DNA plasmids encoding one or more antigen(s) are injected into patients, so that the cells may directly produce said antigen(s), which confer(s) a protective immunological response.

However, the yield of DNA plasmid production is currently a bottleneck for the development of this new therapeutic methodology and the demand will expand in the future. The lysis of the bacterial cell is a key step in the production process of high qualitative amounts of plasmids. This step comes along with large volumes of lysis buffers and generates costly downstream processes for the separation and purification of the plasmid DNA from other cell debris and genomic DNA. For the reasons above, solutions to increase plasmid DNA release upon cell lysis has drawn constant attention of the industry for decades.

To date, most of the research efforts aiming at the optimization of cell lysis have been so far focused on the lysis protocol. Surprisingly, researchers have not been focused, to the knowledge of the applicants, on modifying the bacterial cells in order to improve bacterial lysis sensitivity.

Numerous reports from academic researchers were focused on unraveling the structure-function relationship of the components of the bacterial envelop components (see, e.g., Asmar et al.; PLOS Biology; 2017; Vol. 15(12):e2004303). It is known for decades that Gram-negative bacteria, such as E. coli, contain an envelope that protects the cell from lysis upon osmotic shocks. The envelope is delimited by the cytoplasmic membrane (CM) or inner membrane (IM), which is in contact with the cytoplasm, and the outer membrane (OM), which constitutes the interface with the environment. The CM/IM and the OM are separated by the periplasm, a compartment that contains the peptidoglycan (PG), a single-layer polymer of glycan strands crosslinked by short peptides. In E. coli, tethering the OM to the PG is carried out by the Lpp protein. This protein, which is anchored in the OM via its lipidated N-terminus and attached to the short peptide contained in the PG via its C-terminal lysine. Lpp provides the only covalent connection between the two structures that is mediated by three periplasmic enzymes: YbiS (also referred to as LdtB), YcfS (also referred to as LdtC) and ErfK (also referred to as LdtA). Two additional OM proteins participate in the OM-PG connection through ionic interactions. One is the lipoprotein Pal that belongs to the Tol-Pal constriction apparatus. The lipoprotein Pal interacts independently with TolA, TolB and OmpA (see Cascales et al.; Molecular Microbiology; 2004, Vol. 51(3):873-885). The other one is the β-barrel OmpA protein that extends inside the periplasm through a soluble domain. Both proteins interact non-covalently with the PG through their periplasmic domains. Sonntag et al. (Journal of Bacteriology; 1978; Vol. 136(1):280-285) characterized an E. coli strain with a double deletion mutation in lpp and ompA, in term of morphology, growth needs for electrolytes, sensitivity to hydrophobic antibiotics and detergents, topology of the outer membrane by electron microscopy. In addition, Park et al. (The FASEB Journal; 2012; Vol. 26:219-228) identified the mechanism of anchoring of OmpA to the cell wall peptidoglycan.

Interest in the state of the art was prominent for the production and purification of secreted proteins or proteins that are targeted in the periplasmic compartment before being released in the extracellular medium (for a review on patents, see Yoon et al.; Recent patents on biotechnology; 2010; Vol. 4:23-29). For example, Chen et al. (Microbial Biotechnology; 2014; Vol. 7:360-370) disclosed the construction of leaky strains for the extracellular production of target proteins, in which the genes mrcA, mrcB, pal and lpp from E. coli were knocked out. WO2014044728 disclosed methods for preparing outer membrane vesicles (OMV) comprising heterologous protein that is free in the lumen. The mechanism of production of outer membrane vesicles were disclosed in Schwechheimer et al. (Biochemistry; 2013; Vol. 52:3031-3040; and BMC Microbiology; 2014; Vol. 14:324-335). WO2016183531 disclosed genetically engineered bacteria with mutations of one or more genes encoding a protein that tethers the outer membrane to the peptidoglycan skeleton, and methods to treat hyper-phenylalaninemia.

Leaky mutants of E. coli having mutations in genes such as omp, tol, excC, excD, lpp, env and lky, have been reported to be of special interest for the release of such periplasmic proteins into the extracellular medium (see Kleiner-Grote et al.; Engineering in Life Sciences; 2018, Vol. 18:532-550).

Finally, WO2016210373 disclosed that recombinant bacterial cells may be programmed to express a heterologous gene in response to an exogenous environmental signal and ultimately express a toxin which kills the recombinant bacterial cells. This strategy may be used to treat diseases and disorders.

Therefore, there is a need to identify bacterial strains, e.g. E. coli strains, that, while preserving the capacity of proliferation, would be oversensitive to bacterial lysis and hence allow improving the yield in the production of extra-genomic nucleic acid molecules and/or recombinant polypeptides, in particular recombinant cytoplasmic polypeptides.

### SUMMARY

A first aspect of the invention relates to a genetically modified Escherichia coli bacterium comprising at least two mutated genes encoding proteins involved in the envelope integrity, said bacterium having an altered envelop integrity and being oversensitive to bacterial lysis as compared to a bacterium with unaltered envelop integrity, wherein at least one mutated gene is ompA, and/or a homologue thereof, and at least one mutated gene is a gene involved in Lpp functionality, with the proviso that the bacterium does not comprise simultaneously a complete deletion of the ompA gene and a complete deletion of the lpp gene.

In some embodiments, the at least one gene involved in Lpp functionality is selected in the group comprising or consisting of lpp, ybiS, ycfS and erfK genes, and/or homologues thereof, and any combinations thereof.

In certain embodiments, said at least two mutated genes comprise one of the following combinations:
ompA and lpp, and/or a homologue thereof;
ompA and ybiS, and/or ycfS and/or erfK, and/or a homologue thereof;
ompA, lpp, ybiS and erfK, and/or a homologue thereof;
ompA, lpp, ycfS and erfK, and/or a homologue thereof; or,
ompA, lpp, ybiS and ycfS, and/or a homologue thereof.

In some embodiments, the mutated ompA gene comprises a substitution of the codon encoding arginine (R) at position 256 with a codon encoding a neutrally or negatively charged amino acid, preferably glutamic acid (E) or alanine (A); and/or a substitution of the codon encoding aspartic acid (D) at position 241 with a codon encoding a neutrally or positively charged amino acid, preferably asparagine (N); and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

In certain embodiments, the mutation in the lpp gene is selected in the group comprising, or consisting of, a deletion of the codon encoding lysine (K) at position 58; a substitution of the codon encoding arginine (R) at position 57 with a codon encoding another amino acid, preferably a neutrally charged amino acid, more preferably leucine (L); a substitution of the codon encoding lysine (K) at position 58 with a codon encoding an arginine (R); a complete deletion of the lpp gene; and combinations thereof, wherein said positions are defined with respect to the amino acid sequence SEQ ID NO: 6.

In some embodiments, the mutated ybiS gene, ycfS gene and/or erfK gene, and/or a homologue thereof, consist in a deletion of said ybiS, ycfS and/or erfK genes, and/or a homologue thereof, respectively.

In certain embodiments, said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; and a mutation in the lpp gene consisting of the deletion of the codon encoding lysine (K) at position 58, wherein said position is defined with respect to the amino acid sequence SEQ ID NO: 6.

In some embodiments, said bacterium has a deletion of the C-terminal part of the OmpA protein starting at or before the codon encoding aspartic acid (D) at position 241 or arginine (R) at position 256, wherein said position is defined with respect to the amino acid sequence SEQ ID NO: 3; and a mutation in the lpp gene consisting of the deletion of the codon encoding lysine (K) at position 58, wherein said position is defined with respect to the amino acid sequence SEQ ID NO: 6.

In certain embodiments, said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; a complete deletion of the ybiS gene; a complete deletion of the ycfS gene; and complete deletion of the erfK gene.

In some embodiments, said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; a mutation in the lpp gene consisting of the substitution of the codon encoding arginine (R) at position 57 with a codon encoding a leucine (L), wherein said position is defined with respect to the amino acid sequence SEQ ID NO: 6; a deletion of each of the ybiS gene; and a complete deletion of the ycfS gene.

In certain embodiments, said bacterium has a mutation in the ompA gene consisting of a substitution of the codon encoding aspartic acid (D) at position 241 with a codon encoding asparagine (N), wherein said position is defined with respect to the amino acid sequence SEQ ID NO: 3; and a complete deletion of the lpp gene.

In some embodiments, said bacterium further comprises at least one extra-genomic nucleic acid molecule, preferably encoding at least one polypeptide.

In certain embodiments, said extra-genomic nucleic acid molecule is selected in the group comprising or consisting of a plasmid, a cosmid and a bacterial artificial chromosome (BAC).

Another aspect of the invention pertains to the use of a genetically modified E. coli bacterium comprising at least one extra-genomic nucleic acid molecule and comprising at least one mutated gene encoding a protein involved in the envelope integrity, said bacterium having an altered envelop integrity as compared to a bacterium with unaltered envelop integrity, wherein the at least one mutated gene is ompA, and/or a homologue thereof, or a gene involved in Lpp functionality, for the production and the purification of the at least one extra-genomic nucleic acid molecule.

In some embodiments, the at least one extra-genomic nucleic acid molecule is selected in the group comprising or consisting of a plasmid, a cosmid and a bacterial artificial chromosome (BAC).

A further aspect of the invention relates to the use of genetically modified E. coli bacterium comprising at least one extra-genomic nucleic acid molecule encoding at least one polypeptide and comprising at least one mutated gene encoding a protein involved in the envelope integrity, said bacterium having an altered envelop integrity as compared to a bacterium with unaltered envelop integrity, wherein the at least one mutated gene is ompA, and/or a homologue thereof, or a gene involved in Lpp functionality, for the production and the purification of the at least one polypeptide, preferably encoded by the at least one extra-genomic nucleic acid molecule.

In certain embodiments, the at least one polypeptide is at least one cytoplasmic polypeptide.

In some embodiments, the at least mutated ompA gene consists of a substitution of the codon encoding arginine (R) at position 256 with a codon encoding a neutrally or negatively charged amino acid, preferably glutamic acid (E) or alanine (A); and/or a substitution of the codon encoding aspartic acid (D) at position 241 with a codon encoding a neutrally or positively charged amino acid, preferably asparagine (N); and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

In certain embodiments, the at least mutated gene involved in Lpp functionality consists of a mutation in the lpp gene consisting of the deletion of the codon encoding lysine (K) at position 58, wherein said position being defined with respect to the amino acid sequence SEQ ID NO: 6, or the complete deletion of ybiS gene, and/or the complete deletion of the ycfS gene and/or the complete deletion of the erfK gene.

In some embodiments, the bacterium comprises at least two mutated genes encoding proteins involved in the envelope integrity, and wherein at least one mutated gene is ompA, and/or a homologue thereof, and at least one mutated gene is a gene involved in Lpp functionality.

In certain embodiments, the bacterium does not comprise simultaneously a complete deletion of the ompA gene and a complete deletion of the lpp gene.

In some embodiments, the bacterium is as defined in the instant invention.

In a still further aspect, the invention pertains to a method for the production and the purification of at least one extra-genomic nucleic acid molecule comprising the steps of:
a) culturing genetically modified E. coli bacteria comprising at least one mutated gene encoding a protein involved in the envelope integrity, said bacteria having an altered envelop integrity and being oversensitive to bacterial lysis as compared to a bacterium with unaltered envelop integrity, wherein the at least one mutated gene is ompA, and/or a homologue thereof, or a gene involved in Lpp functionality, said bacteria comprising at least one extra-genomic nucleic acid molecule, so as to amplify the at least extra-genomic nucleic acid molecule;
b) lysing the bacteria obtained at step a), preferably by chemical lysis, so as to obtain a lysis mixture; and,
c) purifying said amplified extra-genomic nucleic acid molecule from the lysis mixture obtained at step b).

In certain embodiments, the at least one extra-genomic nucleic acid molecule is selected in the group comprising or consisting of a plasmid, a cosmid and a bacterial artificial chromosome (BAC).

Another aspect of the instant invention relates to a method for the production and the purification of at least one polypeptide, preferably encoded by an extra-genomic nucleic acid molecule, comprising the steps of:
a) culturing genetically modified E. coli bacteria comprising at least one mutated gene encoding a protein involved in the envelope integrity, said bacteria having an altered envelop integrity and being oversensitive to bacterial lysis as compared to a bacterium with unaltered envelop integrity, wherein the at least one mutated gene is ompA, and/or a homologue thereof, or a gene involved in Lpp functionality, said bacteria preferably comprising at least one extra-genomic nucleic acid molecule encoding the at least one polypeptide, so as to synthesize the at least one polypeptide;
b) lysing the bacteria obtained at step a), so as to obtain a lysis mixture; and,
c) purifying said at least one polypeptide from a lysis mixture obtained at step b).

In some embodiments, the at least one polypeptide is one cytoplasmic polypeptide.

In certain embodiments, the at least mutated ompA gene consists of a substitution of the codon encoding arginine (R) at position 256 with a codon encoding a neutrally or negatively charged amino acid, preferably glutamic acid (E) or alanine (A); and/or a substitution of the codon encoding aspartic acid (D) at position 241 with a codon encoding a neutrally or positively charged amino acid, preferably asparagine (N); and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

In some embodiments, the at least mutated gene involved in Lpp functionality consists of a mutation in the lpp gene consisting of the deletion of the codon encoding lysine (K) at position 58, wherein said position being defined with respect to the amino acid sequence SEQ ID NO: 6, or the complete deletion of ybiS gene, and/or the complete deletion of the ycfS gene and/or the complete deletion of the erfK gene.

In certain embodiments, the bacterium comprises at least two mutated genes encoding proteins involved in the envelope integrity, and wherein at least one mutated gene is ompA, and/or a homologue thereof, and at least one mutated gene is a gene involved in Lpp functionality.

In some embodiments, the bacterium does not comprise simultaneously a complete deletion of the ompA gene and a complete deletion of the lpp gene.

In certain embodiments, the bacterium is as defined in the instant disclosure.

One aspect of the instant invention relates to a kit comprising (i) a genetically modified E. coli bacterium comprising at least one mutated gene encoding a protein involved in the envelope integrity, said bacterium having an altered envelop integrity and being oversensitive to bacterial lysis as compared to a bacterium with unaltered envelop integrity, wherein the at least one mutated gene is ompA, and/or a homologue thereof, or a gene involved in Lpp functionality; and (ii) means to transform said bacterium with an extra-genomic nucleic acid molecule.

In some embodiments, the genetically modified E. coli bacterium comprises at least two mutated genes encoding proteins involved in the envelope integrity and wherein at least one mutated gene is ompA, and/or a homologue thereof, and at least one mutated gene is a gene involved in Lpp functionality.

### Definitions

In the present invention, the following terms have the following meanings:

“About” preceding a figure encompasses the range spanning plus or minus 10% of the value of said figure. It is to be understood that the value to which the term “about” refers is itself also specifically, and preferably, disclosed.

“At least one” includes 1, 2, 3, 4, 5, 6, 7, 8, 9, 10 or more.

“At least two” includes 2, 3, 4, 5, 6, 7, 8, 9, 10 or more.

“Bacterium” as used herein, refers to one bacterial cell. Therefore, the term “bacteria” refers to a population of bacterial cells.

“Gene involved in Lpp functionality” refers to any gene which expression results in a physiologically functional Lpp polypeptide. It is to be understood herein that “a physiologically functional Lpp polypeptide” refers to a Lpp polypeptide that is located in the bacterial envelop and participate in the envelop integrity in a gram-negative bacterium, in particular E. coli.

“Extra-genomic nucleic acid molecule”, as used herein, refers to a deoxyribonucleic acid (DNA) molecule that is present in a bacterium but that is not integrated in, or part of, the bacterial chromosome. Extra-genomic nucleic acids may be linear or circular. Extra-genomic nucleic acids may comprise a selectable marker such as for instance a sequence conferring to the host bacteria resistance to an antibiotic, or a lacZ sequence encoding a β-galactosidase for blue/white selection. Extra-genomic nucleic acids typically comprise sequences allowing their replication (e.g. origins of replication pMB1, ColE1 or f1) and the regulation of the number of copies per cell, such as for instance the repE gene, or the rop gene. Extra-genomic nucleic acids may comprise promoter sequences allowing the expression of downstream sequences such as for example the T7 promoter or the SP6 promoter. The expression “extra-genomic nucleic acid” includes, but it not limited to, plasmids, cosmids and bacterial artificial chromosomes (BAC). A “plasmid” refers to a small extra-genomic DNA molecule, most commonly found as circular double stranded DNA molecules that may be used as a cloning vector in molecular biology, to make and/or modify copies of DNA fragments up to about 15 kb (i.e. 15,000 base pairs). Plasmids may also be used as expression vectors to produce large amounts of proteins of interest encoded by a nucleic acid sequence found in the plasmid downstream of a promoter sequence. The term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid. Cosmids are typically used as cloning vector for DNA fragments ranging in size from about 32 to 52 kb. “Bacterial artificial chromosome” or “BAC” refers to an extra-genomic nucleic acid molecule based on a functional fertility plasmid that allows the even partition of said extra-genomic DNA molecules after division of the bacterial cell. BACs are typically used as cloning vector for DNA fragment ranging in size from about 150 to 350 kb.

“Gene” as used herein, refers to a nucleic acid sequence associated to a particular function. Examples of final products encoded by a gene are RNAs and proteins.

“Genetically modified” is used herein in reference to a microorganism, in particular a gram-negative bacterium in the context of the invention, comprising at least one mutation that has been actively generated and/or selected for.

“Gram-negative”, as used herein, refers to a bacterium characterized by its cellular envelope, which is composed of a thin peptidoglycan wall sandwiched between an inner cytoplasmic membrane and an outer membrane. Gram negative bacteria may be easily identified by Gram staining, developed by the Danish bacteriologist Hans Christian Gram. Whereas Gram positive bacteria, which have a cytoplasmic membrane surrounded by a thick peptidoglycan wall are stained in purple after Gram staining, Gram negative bacteria are stained in pink/red.

“Identity”, when used in a relationship between the sequences of two or more polypeptides or of two or more nucleic acid sequences, refers to the degree of sequence relatedness between polypeptides or nucleic acid sequences (respectively), as determined by the number of matches between strings of two or more amino acid residues or of two or more nucleotides, respectively. “Identity” measures the percent of identical matches between the smaller of two or more sequences with gap alignments (if any) addressed by a particular mathematical model or computer program (i.e., “algorithms”). Identity of related polypeptides or nucleic acid sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 2, 387 (1984); Genetics Computer Group, University of Wisconsin, Madison, Wis.), BLASTP, BLASTN, TBLASTN and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity. In one embodiment, the term identity is measured over the entire length of the sequence to which it refers.

“Mutation” is used herein in reference to a gene and refers to an alteration of the nucleic acid sequence of said gene. A mutation may comprise a substitution of one or more, nucleotide(s) in the gene's nucleic acid sequence, such as transitions, that exchange a purine for a purine (A↔G) or a pyrimidine for a pyrimidine, (C↔T), or transversions, that exchange a purine for a pyrimidine or a pyrimidine for a purine (C/T↔A/G). A mutation may also comprise a deletion or an insertion of one or more nucleotide(s) in the nucleic acid sequence of the gene. A mutation may concern the sequence encoding the final gene product (RNA or protein). In cases in which the mutation affects the coding sequence of a polypeptide and leads to a change in the amino acid sequence of said corresponding polypeptide, the mutation may be defined by the modification in the amino acid sequence of said polypeptide. The skilled artisan is able to identify the codon(s) of interest in the nucleic acid sequence encoding said polypeptide and design, using the genetic code, the appropriate modification(s) in the nucleic acid sequence, to obtain following transcription and translation, the desired mutated polypeptide sequence. The term mutation, as used herein, also includes deletions in the nucleic acid sequence of a gene encompassing the entire sequence encoding the final gene product (RNA or polypeptide). The latter type of mutation is referred to herein as a “complete deletion”.

In one embodiment, the term mutation, when used in the sentence “an organism comprising mutation(s) in the gene x” herein signifies that any copy (or copies) of the gene x, either on the bacterial chromosome or on an extra-genomic nucleic acid molecule, present in said microorganism comprise said mutation(s). As used herein, a mutation may affect the transcription of a mutated gene into the corresponding mRNA; may affect the translation of the mRNA into the corresponding polypeptide. In one embodiment, the nucleic acid sequence with the mutation(s) is inherited by the progeny of the microorganism, such as it is the case for nucleic acid sequences found in the bacterial chromosome of a bacteria. In one embodiment, the nucleic acid sequence with the mutation(s) is found in the extra-chromosomal DNA of a bacteria.

“Homologue” may refer to a polypeptide or a nucleic acid sequence that shares from 30% to 99.99% sequence identity with a reference polypeptide or a nucleic acid sequence (also referred to as a “structural homologue”) and/or that shares identical or similar biological function with the reference polypeptide or a nucleic acid sequence. Sequence identity may be determined as explained above (also referred to as a “functional homologue”). The biological function may be assessed by any suitable method known in the art, or a method derived therefrom. In some embodiments, the homologue is a structural homologue. In alternative embodiments, the homologue is a functional homologue.

“Amino acid conservation” when used in a relationship between the sequences of two or more polypeptides or of two or more nucleic acid sequences, refers to the degree of amino acid sequence relatedness between a given region in said polypeptides or nucleic acid sequences. For example, amino acid conservation for a given amino acid position may refer to either a unique amino acid or to a related amino acid. Illustratively, hydrophobic amino acid such as Leu, Ile, Val may be considered as related amino acids.

It is the same with positively charged amino acids Lys, Arg, His and to negatively charged amino acid such as Glu and Asp. In some embodiments, conserved amino acids within a region from two or more polypeptides may be referred to as a “consensus sequence”.

“Concentration” or “concentrate” may refer to the action of locally accumulating a target of interest.

“Purification” or “purify” may refer to the action of obtaining a pure or substantially pure compound of interest, from a mixture of compounds.

“Polypeptide” refers to a linear polymer of at least 50 amino acids linked together by peptide bonds. In some embodiments, a polypeptide refers to a cytoplasmic polypeptide, and/or to a non-secreted polypeptide, namely a polypeptide destined to remain in the cytoplasm upon synthesis. In some embodiments, the cytoplasmic polypeptide is folded, i.e., has acquired a 2-dimensional or a 3-dimensional structure.

“Protein” refers to a functional entity formed of one or more peptides or polypeptides, and optionally of non-polypeptides cofactors. In some embodiments, a protein refers to a cytoplasmic protein, or to a non-secreted protein, namely a protein destined to remain in the cytoplasm upon synthesis. In some embodiments, the cytoplasmic protein is folded, i.e., has acquired a 2-dimensional or a 3-dimensional structure.

“Bacterial lysis” refers to the release of soluble material from the cell, including from the cytoplasm.

### DETAILED DESCRIPTION

The present invention relates to a genetically modified gram-negative bacterium, namely Escherichia coli, having an altered envelope integrity and being oversensitive to bacterial lysis, as compared to a bacterium with unaltered envelop integrity. The inventors have engineered bacterial strains derived from E. coli that can sustain growth in suitable culture conditions and provide high yield of plasmids or plasmid-encoded polypeptides. In addition, plasmids or plasmid-encoded polypeptides, in particular cytoplasmic polypeptides, may be recovered upon bacterial lysis of the engineered E. coli strains with high yield. Finally, the experimental data provided herein suggest that high yield of cytoplasmic molecules, such as, plasmids or plasmid-encoded polypeptides (recombinant polypeptides) may be recovered with reduced contamination of genomic nucleic acids, bacterial cytoplasmic proteins and/or cell debris.

A first aspect of the invention relates to a genetically modified gram-negative bacterium comprising at least two mutated genes encoding proteins involved in the envelope integrity, said bacterium having an altered envelop integrity and being oversensitive to bacterial lysis as compared to a bacterium with unaltered envelop integrity.

As used herein, “at least two” encompasses 2, 3, 4, 5, 6, 7, 8, 9, 10 or more.

In some embodiments, said bacterium is selected in a group comprising a bacterium of the Proteobacterium phylum, preferably of the Gamma Proteobacteria class, preferably of the Enterobacteriaceae family, preferably of the genus Escherichia, more preferably of the species Escherichia coli.

In some embodiments, the genetically modified gram-negative bacterium of the invention is a non-pathogenic bacterium. As used herein, the expression “non-pathogenic” refers to a bacterium that does not harm a living organism, in particular an animal organism, preferably a human organism, upon contacting said organism. In particular, the expression “does not harm” is intended to mean that the bacterium does not result in an infection, a disorder or a disease.

In one embodiment, the genetically modified gram-negative bacterium of the invention is selected in a group comprising a bacterium of the Proteobacteria phylum. As used herein, a bacterium of the Proteobacteria phylum includes a bacterium of the Alpha Proteobacteria class, the Beta Proteobacteria class, the Gamma Proteobacteria class, the Delta Proteobacteria class, the Epsilon Proteobacteria class and the Zeta Proteobacteria class.

In one embodiment, the genetically modified gram-negative bacterium of the invention is of the Gamma Proteobacteria class. As used herein, a bacterium of the Gamma Proteobacteria class includes a bacterium of the Acidithiobacillaceae, Aeromonadaceae, Alteromonadaceae, Cardiobacteriaceae, Chromatiaceae, Enterobacteriaceae, Legionellae, Methylococcaceae, Oceanospirillaceae, Pasteurellaceae, Pseudomonadaceae, Thiotrichaea, Vibrionaceae and Xanthomonadaceae family.

In one embodiment, the genetically modified gram-negative bacterium of the invention is of the Enterobacteriaceae family. The term “Enterobacteriaceae”, as used herein, refers to a family of gram-negative bacteria that includes over 50 genera and over 200 species. Within the scope of the invention, non-limitative examples of bacteria of the Enterobacteriaceae family include bacteria of the genus Citrobacter, Enterobacter, Escherichia, Klebsiella, Morganella, Proteus, Providencia, Salmonella, Serratia, Shigella and Yersinia.

In one embodiment, the genetically modified gram-negative bacterium of the invention is of the genus Escherichia. Within the scope of the invention, non-limitative examples of bacteria of the genus Escherichia include bacteria of the species E. adecarboxylata, E. albertii, E. blattae, E. coli, E. fergusonii, E. hermannii, and E. vulneris.

In one embodiment, the genetically modified gram-negative bacterium of the invention is of the species Escherichia coli. As used herein, “Escherichia coli”, also referred to as “E. coli”, refers to a bacterial species that is naturally found in the intestinal flora of many mammal individuals, in particular human individuals. Bacterium belonging to the species E. coli are also used in industrial fermentation processes to synthesize various products, in particular in the context of the invention to synthesize biological molecules, such as e.g. polypeptides and nucleic acid molecules.

Another aspect of the invention pertains to a genetically modified E. coli bacterium comprising at least two mutated genes encoding proteins involved in the envelope integrity, said bacterium having an altered envelop integrity and being oversensitive to bacterial lysis as compared to a bacterium with unaltered envelop integrity, wherein at least one mutated gene is ompA, and/or a homologue thereof, and at least one mutated gene is a gene involved in Lpp functionality, with the proviso that the bacterium does not comprise simultaneously a complete deletion of the ompA gene and a complete deletion of the lpp gene.

As used herein, “gene involved in Lpp functionality” refers to any gene which expression results in a physiologically functional Lpp polypeptide. In some embodiment, a gene involved in Lpp functionality includes the lpp gene itself, which encodes the Lpp polypeptide. In some embodiments, a gene involved in Lpp functionality includes any one of genes ybiS, ycfS and erfK, which encodes respectively the YbiS, YcfS and ErfK polypeptide.

In some embodiments, the at least one gene involved in Lpp functionality is selected in the group comprising or consisting of lpp, ybiS, ycfS and erfK genes, and/or homologues thereof, and any combinations thereof.

In one embodiment, the E. coli bacterium strain is selected in the non-limiting group comprising the BL21(DE3) strain, the DH5-Alpha strain, the DH10B strain, the INV110 strain, the Mach1 strain, the MG1655 strain, the Rosetta® strain and the TOP10 strain.

In practice, the BL21(DE3) strain has the following genotype: F⁻ ompT hsdS_{B} (r_{B}⁻, m_{B}⁻) gal dcm (DE3); the DH5-Alpha strain has the following genotype: F⁻ φ80lacZΔM15 Δ(lacZYA-argF)U169 recA1 endA1 hsdR17(r_{K}⁻,m_{K}⁺) phoA supE44 λ⁻ thi-1 gyrA96 relA1; the DH10B strain has the following genotype: F⁻ mcrA Δ(mrr-hsdRMS-mcrBC) φ80lacZΔM15 ΔlacX74 recA1 endA1 araD139 Δ(ara-leu)7697 galU galK λ⁻ rpsL(Str^{R}) nupG; the INV110 strain has the following genotype: F′ [traD36 proAB lacI^{q} lacZΔM15] rpsL (Str^{R}) thr leu endA thi-1 lacY galK galT ara tonA tsx dam dcm supE44 Δ(lac-proAB) Δ(mcrC-mrr)102::Tn10(Tet^{R}); the Mach1 strain has the following genotype: F⁻ φ80lacZΔM15 ΔlacX74 hsdR(r_{K}⁻, m_{K}⁺) ΔrecA1398 endA1 tonA; the MG1655 strain has the following genotype: F⁻ λ⁻ ilvG⁻ rfb-50 rph-1; the Rosetta® strain has the following genotype: F⁻ ompT hsdSB(r_{B}⁻ m_{B}⁻) gal dcm (DE3) pRARE (Cam^{R}); the TOP10 strain has the following genotype: F⁻ mcrA Δ(mrr-hsdRMS-mcrBC) φ80lacZΔM15 ΔlacX74 recA1 araD139 Δ(ara-leu)7697 galU galK λ⁻ rpsL(Str^{R}) endA1 nupG.

Within the scope of the instant invention, the expression “protein involved in the envelope integrity” is meant to refer to a protein being known as a structural element of the bacterial envelope of a gram-negative bacterium and/or as an element participating in the synthesis and/or the maintenance of the bacterial envelope. Examples of proteins involved in the envelope integrity include, but are not limited to periplasmic proteins, outer membrane proteins, proteins participating in the attachment of the inner membrane to the periplasmic peptidoglycan, in the attachment of the periplasmic peptidoglycan to the outer membrane, and/or in the attachment of the inner membrane to the outer membrane. Illustratively, proteins involved in the envelope integrity according to the invention include, but are not limited to, the Outer membrane protein A (OmpA) and/or a homologue thereof; the major outer membrane pro-lipoprotein Lpp (Lpp) and/or a homologue thereof; proteins of the trans-envelope Tol-Pal complex such as the peptidoglycan-associated lipoprotein (Pal) and the L,D-transpeptidases that are responsible of the cross-linking of Lpp to the short peptide backbone present in periplasmic peptidoglycans, such as YbiS, YcfS and ErfK.

In some embodiment, the mutations in the genes encoding proteins involved in the envelope integrity are mutations that alter the envelope integrity. In certain embodiments, the alteration of the integrity of the envelope integrity is an impairment or a disruption of the envelop integrity.

Techniques to evaluate the integrity of the bacterial envelope of a gram-negative bacterium are known to the skilled artisan and include, without being limited to, testing permeability to labelled compound of know size (such as for example labelled dextrans), resistance to an osmotic shock. In some embodiments, the integrity of the bacterial envelope may be evaluated by assessing the interactions between the peptidoglycan and interacting proteins, e.g. as disclosed in Ishikawa et al. (Mol Microbiol. 2016 August; 101(3):394-410).

In some embodiment, the mutation in each of the at least two genes encoding a protein involved in the envelope integrity is a mutation that disrupts the attachment of the inner membrane to the periplasmic peptidoglycan, the attachment of the periplasmic peptidoglycan to the outer membrane or the attachment of the inner membrane to the outer membrane.

Techniques to evaluate the attachment of the outer membrane to periplasmic peptidoglycans are known to the skilled artisan and include, without being limited to, observation of the thickness of the periplasm by electron microscopy, microscopic observation of outer membrane blebbing, co-immunoprecipitation of interacting proteins, crosslinking of interacting proteins.

In some embodiments, said at least two mutated genes are selected in the group comprising ompA, lpp, pal, ybiS, ycfS and erfK genes, and/or homologues thereof, and wherein at least one of the at least two mutated genes is the ompA gene or the lpp gene, or a homologue thereof.

In certain embodiments, said at least two mutated genes comprise one of the following combinations:
ompA and lpp, and/or a homologue thereof;
lpp and pal, and/or a homologue thereof;
ompA and pal, and/or a homologue thereof;
ompA, ybiS, ycfS and erfK, and/or a homologue thereof;
ompA, lpp, ybiS and erfK, and/or a homologue thereof;
ompA, lpp, ycfS and erfK, and/or a homologue thereof; or,
ompA. lpp, ybiS and ycfS, and/or a homologue thereof.

In certain embodiments, said at least two mutated genes comprise one of the following combinations:
ompA and lpp, and/or a homologue thereof;
lpp, and pal, and/or a homologue thereof;
ompA, and pal, and/or a homologue thereof;
ompA, ybiS, ycfS and erfK, and/or a homologue thereof;
ompA, lpp, ybiS and erfK, and/or a homologue thereof;
ompA, lpp, ycfS and erfK, and/or a homologue thereof; or,
ompA, lpp, ybiS and ycfS, and/or a homologue thereof.

In some embodiments, at least two mutated genes comprise one of the following combinations:
ompA and lpp, and/or a homologue thereof;
ompA and ybiS, and/or ycfS and/or erfK, and/or a homologue thereof;
ompA, lpp, ybiS and erfK, and/or a homologue thereof;
ompA, lpp, ycfS and erfK, and/or a homologue thereof; or,
ompA, lpp, ybiS and ycfS, and/or a homologue thereof.

Techniques to generate a mutation in a bacterial gene are known to the skilled artisan and include, without being limited to, phage transduction, chemical mutagenesis, homologous recombination, genome editing with CRISPR-cas9, zinc-finger domain-nucleases fusions.

In one embodiment, the genetically modified gram-negative bacterium of the invention, in particular E. coli bacterium, comprises at least one mutation in the ompA gene, and/or a homologue thereof.

The ompA gene is naturally found in the genome of E. coli in which it encodes the outer membrane protein A. The OmpA protein spans across the outer membrane of the bacterial envelope of gram-negative bacteria by the means of its N-terminal β-barrel. The soluble C-terminal portion of the protein extends inside the periplasm and interacts non-covalently with the periplasmic peptidoglycan. It is understood that the ompA gene encodes a protein involved in the envelope integrity of the gram-negative bacterium according to the invention. More precisely, the ompA gene encodes a protein involved in the attachment of the outer membrane of the bacterial envelope to the periplasmic peptidoglycan.

In certain embodiments, the ompA gene refers to a nucleic acid with the EcoCyc accession number EG10669. In some embodiments, the ompA gene is represented by a nucleic acid sequence having at least 75% nucleic acid sequence identity to SEQ ID NO: 1. Within the scope of the invention, the expression “at least 75% nucleic acid identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% nucleic acid identity.

The level of identity of 2 nucleic acid sequences may be performed by using any one of the known algorithms available from the state of the art.

Illustratively, the nucleic acid identity percentage may be determined using the CLUSTAL W software (version 1.83) the parameters being set as follows:
for slow/accurate alignments: (1) Gap Open Penalty: 15; (2) Gap Extension Penalty: 6.66; (3) Weight matrix: IUB;
for fast/approximate alignments: (4) K-tuple (word) size: 2; (5) Gap Penalty: 5; (6) No. of top diagonals: 5; (7) Window size: 4; (8) Scoring Method: PERCENT.

In some embodiments, the ompA gene is represented by a nucleic acid sequence having at least 80%, preferably at least 90%, more preferably at least 95% nucleic acid sequence identity to SEQ ID NO: 1.

In one embodiment, the ompA gene is represented by a nucleic acid sequence consisting of SEQ ID NO: 1.

In certain embodiments, the OmpA protein refers to a preprotein with the UniProtKB accession number P0A910. In some embodiments, the OmpA preprotein is represented by an amino acid sequence having at least 75% amino acid sequence identity to SEQ ID NO: 2. Within the scope of the invention, the expression “at least 75% amino acid identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% amino acid identity.

Illustratively, the amino acid identity percentage may also be determined using the CLUSTAL W software (version 1.83) the parameters being set as follows:
for slow/accurate alignments: (1) Gap Open Penalty: 10.00; (2) Gap Extension Penalty:0.1; (3) Protein weight matrix: BLOSUM;
for fast/approximate alignments: (4) Gap penalty: 3; (5) K-tuple (word) size: 1; (6) No. of top diagonals: 5; (7) Window size: 5; (8) Scoring Method: PERCENT.

In some embodiments, the OmpA preprotein is represented by an amino acid sequence having at least 80%, preferably at least 90%, more preferably at least 95% amino acid sequence identity to SEQ ID NO: 2.

In one embodiment, the OmpA preprotein is represented by an amino acid sequence consisting of SEQ ID NO: 2.

In one embodiment, the mutation in the ompA gene is a mutation promoting the disruption of the binding of OmpA to the periplasmic peptidoglycan. As used herein, the expression “disrupting the binding of OmpA to the periplasmic peptidoglycan” refers to a level of covalent binding of OmpA to the periplasmic peptidoglycan reaching at most about 75% of the level of covalent binding observed in bacterium with unaltered envelop integrity.

Within the scope of the invention, the expression “at most about 75%” encompasses about 75%, 70%, 65%, 60%, 55%, 50%, 45%, 40%, 35%, 30%, 25%, 20%, 15%, 10%, 5%, 1%, 0.5% and 0.1%.

Techniques to evaluate the binding of OmpA to the periplasmic peptidoglycan are known to the skilled artisan and include, without being limited to, co-separation-based techniques, such as for example co-immunoprecipitation, GST pull-down and the like; fluorescence-based assays, such as for example FRET, BiFC and the like; surface plasmon resonance-based assay; genetic reporter-based assays such as for example, yeast-2-hybrid, phage display and the like.

During the natural addressing of the OmpA preprotein to the outer membrane, OmpA preprotein is cleaved as to release its 21 aa-long N-terminal signal peptide and the 325 aa-long mature protein. In practice, the position of the mutations in the ompA gene may be defined with respect to the corresponding codon encoding the amino acid at a given position, taking as a reference the mature OmpA protein of amino acid sequence SEQ ID NO: 3.

In one embodiment, the OmpA mature protein is represented by an amino acid sequence having at least 75%, preferably at least 80%, preferably at least 90%, more preferably at least 95% amino acid sequence identity to SEQ ID NO: 3. In one embodiment, the OmpA mature protein is represented by an amino acid sequence consisting of SEQ ID NO: 3.

In some embodiments, the mutated ompA gene comprises a substitution of the codon encoding arginine (R) at position 256 with a codon encoding a neutrally or negatively charged amino acid, preferably glutamic acid (E) or alanine (A); and/or a substitution of the codon encoding aspartic acid (D) at position 241 with a codon encoding a neutrally or positively charged amino acid, preferably asparagine (N); and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

As used herein, the expression “neutrally charged amino acid” refers to an amino acid selected in the group comprising, or consisting of, alanine (A), asparagine (N), cysteine (C), glutamine (Q), glycine (G), isoleucine (I), leucine (L), methionine (M), phenylalanine (F), proline (P), serine (S), threonine (T), tryptophan (W), tyrosine (Y) and valine (V). As used herein, the expression “negatively charged amino acid” refers to an amino acid selected in the group comprising, or consisting of, glutamic acid (E) and aspartic acid (D). As used herein, the expression “positively charged amino acid” refers to an amino acid selected in the group comprising, or consisting of, arginine (R), histidine (H) and lysine (K).

In some embodiments, a deletion of the C-terminal part of the OmpA protein consists of a deletion starting at or before the codon encoding aspartic acid (D) at position 241, wherein said position is defined with respect to the amino acid sequence SEQ ID NO: 3.

In certain embodiments, a deletion of the C-terminal part of the OmpA protein consists of a deletion starting at or before the codon encoding arginine (R) at position 256, wherein said position is defined with respect to the amino acid sequence SEQ ID NO: 3.

In some embodiments, the homologue(s) of the ompA gene is/are selected in a group consisting of the yfiB gene and the yiaD gene. In certain embodiments, the homologue(s) of the OmpA protein is/are selected in a group consisting of the YfiB protein and the YiaD protein.

In practice, the yfiB gene refers to a nucleic acid with the EcoCyc accession number EG11152. In some embodiments, the yfiB gene is represented by a nucleic acid sequence having at least 75% nucleic acid sequence identity to SEQ ID NO: 16. In some embodiments, the yfiB gene is represented by a nucleic acid sequence having at least 80%, preferably at least 90%, more preferably at least 95% nucleic acid sequence identity to SEQ ID NO: 16. In one embodiment, the yfiB gene is represented by a nucleic acid sequence consisting of SEQ ID NO: 16.

In certain embodiments, the YfiB protein refers to a polypeptide with the UniProtKB accession number P07021. In some embodiments, the YfiB protein is represented by an amino acid sequence having at least 75% amino acid sequence identity to SEQ ID NO: 17. In some embodiments, the YfiB protein is represented by an amino acid sequence having at least 80%, preferably at least 90%, more preferably at least 95% amino acid sequence identity to SEQ ID NO: 17. In one embodiment, the YfiB protein is represented by an amino acid sequence consisting of SEQ ID NO: 17.

Illustratively, the region from amino acid 43 to amino acid 160 of YfiB is conserved with OmpA. Having identified the conserved amino acids between YfiB and OmpA, one may retrieve the corresponding mutations from the OmpA protein in the YfiB protein, and vice-versa, when applicable.

In practice, the yiaD gene refers to a nucleic acid with the EcoCyc accession number EG12271. In some embodiments, the yiaD gene is represented by a nucleic acid sequence having at least 75% nucleic acid sequence identity to SEQ ID NO: 18. In some embodiments, the yiaD gene is represented by a nucleic acid sequence having at least 80%, preferably at least 90%, more preferably at least 95% nucleic acid sequence identity to SEQ ID NO: 18. In one embodiment, the yiaD gene is represented by a nucleic acid sequence consisting of SEQ ID NO: 18.

In certain embodiments, the YiaD protein refers to a polypeptide with the UniProtKB accession number P37665. In some embodiments, the YiaD protein is represented by an amino acid sequence having at least 75% amino acid sequence identity to SEQ ID NO: 19. In some embodiments, the YiaD protein is represented by an amino acid sequence having at least 80%, preferably at least 90%, more preferably at least 95% amino acid sequence identity to SEQ ID NO: 19. In one embodiment, the YiaD protein is represented by an amino acid sequence consisting of SEQ ID NO: 19.

Illustratively, the region from amino acid 103 to amino acid 219 of YiaD is conserved with OmpA. Having identified the conserved amino acids between YiaD and OmpA, one may retrieve the corresponding mutations from the OmpA protein in the YiaD protein, and vice-versa, when applicable.

In one embodiment, the genetically modified gram-negative bacterium of the invention, in particular E. coli bacterium, comprises at least one mutation in the lpp gene, and/or a homologue thereof.

The lpp gene is naturally found in the genome of E. coli in which it encodes the major outer membrane pro-lipoprotein Lpp. The Lpp protein tethers the outer membrane of the bacterial envelope to the periplasmic peptidoglycan. The Lpp protein is anchored via its lipidated N-terminus to the outer membrane and is covalently attached via its C-terminal lysine to the short peptide backbone present in periplasmic peptidoglycan. It is understood that the lpp gene encodes a protein involved in the envelope integrity of the gram-negative bacterium according to the invention. More precisely, the lpp gene encodes a protein involved in the covalent attachment of the outer membrane of the bacterial envelope to the periplasmic peptidoglycan.

In certain embodiments, the lpp gene refers to a nucleic acid with the EcoCyc accession number EG10544. In some embodiments, the lpp gene is represented by a nucleic acid sequence having at least 75% nucleic acid sequence identity to SEQ ID NO: 4.

In some embodiments, the lpp gene is represented by a nucleic acid sequence having at least 80%, preferably at least 90%, more preferably at least 95% nucleic acid sequence identity to SEQ ID NO: 4.

In one embodiment, the lpp gene is represented by a nucleic acid sequence consisting of SEQ ID NO: 4.

In certain embodiments, the Lpp protein refers to a preprotein with the UniProtKB access number P69776. In some embodiments, the Lpp preprotein is represented by an amino acid sequence having at least 75% amino acid sequence identity to SEQ ID NO: 5.

In some embodiments, the Lpp preprotein is represented by an amino acid sequence having at least 80%, preferably at least 90%, more preferably at least 95% amino acid sequence identity to SEQ ID NO: 5.

In one embodiment, the Lpp preprotein is represented by an amino acid sequence consisting of SEQ ID NO: 5.

In one embodiment, the mutation in the lpp gene is a mutation disrupting the binding of Lpp to the periplasmic peptidoglycan. As used herein, the expression “disrupting the binding of Lpp to the periplasmic peptidoglycan” refers to a level of covalent binding of Lpp to the periplasmic peptidoglycan reaching at most about 75% of the level of covalent binding observed in bacterium with unaltered envelop integrity. In some embodiments, the level of covalent binding of Lpp to the periplasmic peptidoglycan may be assessed by the means of an antibody that specifically binds to the Lpp protein.

Techniques to evaluate the binding of Lpp to the periplasmic peptidoglycan are known to the skilled artisan and include similar techniques as the techniques useful for evaluating the binding of OmpA to the periplasmic peptidoglycan. One can also refer to Zhang et al. (J. Biol. Chem. 1992 September; 267(27):19560-4 and 19631-5); Ishikawa et al. (Mol Microbiol. 2016 August; 101(3):394-410); Cowles et al. (Mol. Microbiol. 2011 March; 79(5):1168-81).

Lpp is naturally synthesized as a 78 aa-long preprotein, which is subsequently cleaved during its addressing to the periplasm, as to release a 20 aa-long N-terminal signal peptide and a 58 aa-long mature protein. In practice, the position of the mutations in the lpp gene are defined with respect to the corresponding codon encoding the amino acid at a given position, taking as a reference the mature Lpp protein of amino acid sequence SEQ ID NO: 6.

In one embodiment, the Lpp mature protein is represented by an amino acid sequence having at least 75%, preferably at least 80%, preferably at least 90%, more preferably at least 95% amino acid sequence identity to SEQ ID NO: 6. In one embodiment, the Lpp mature protein is represented by an amino acid sequence consisting of SEQ ID NO: 6.

In one embodiment, the mutation in the lpp gene is selected in the group comprising, or consisting of, a deletion of the codon encoding lysine (K) at position 58; a substitution of the codon encoding arginine (R) at position 57 with a codon encoding another amino acid, preferably a neutrally charged amino acid, more preferably leucine (L); a substitution of the codon encoding lysine (K) at position 58 with a codon encoding an arginine (R); a complete deletion of the lpp gene; and combinations thereof, wherein said positions are defined with respect to the amino acid sequence SEQ ID NO: 6.

In some embodiments, the homologue of the lpp gene is the yqhH gene. In certain embodiments, the homologue of the Lpp protein is the YqhH protein.

In practice, the yqhH gene refers to a nucleic acid with the EcoCyc accession number G7567. In some embodiments, the yqhH gene is represented by a nucleic acid sequence having at least 75% nucleic acid sequence identity to SEQ ID NO: 20. In some embodiments, the yqhH gene is represented by a nucleic acid sequence having at least 80%, preferably at least 90%, more preferably at least 95% nucleic acid sequence identity to SEQ ID NO: 20. In one embodiment, the yqhH gene is represented by a nucleic acid sequence consisting of SEQ ID NO: 20.

In certain embodiments, the YqhH protein refers to a polypeptide with the UniProtKB accession number P65298. In some embodiments, the YqhH protein is represented by an amino acid sequence having at least 75% amino acid sequence identity to SEQ ID NO: 21. In some embodiments, the YqhH protein is represented by an amino acid sequence having at least 80%, preferably at least 90%, more preferably at least 95% amino acid sequence identity to SEQ ID NO: 21. In one embodiment, the YqhH protein is represented by an amino acid sequence consisting of SEQ ID NO: 21.

Illustratively, the region from amino acid 25 to amino acid 71 of YqhH is conserved with Lpp. Having identified the conserved amino acids between YqhH and Lpp, one may retrieve the corresponding mutations from the Lpp protein in the YqhH protein, and vice-versa, when applicable.

In one embodiment, the genetically modified gram-negative bacterium of the invention, in particular E. coli bacterium, comprises at least one mutation in the pal gene.

The pal gene is naturally found in the genome of E. coli in which it encodes the peptidoglycan-associated lipoprotein. The Pal protein is important for maintaining the outer membrane integrity. It is understood that the pal gene encodes a protein involved in the envelope integrity of a gram-negative bacterium according to the invention. More precisely, the pal gene encodes a protein involved in the attachment of the outer membrane of the bacterial envelope to the periplasmic peptidoglycan.

In certain embodiments, the pal gene refers to a nucleic acid with the EcoCyc accession number EG10684. In some embodiments, the pal gene is represented by a nucleic acid sequence having at least 75% nucleic acid sequence identity to SEQ ID NO: 7.

In some embodiments, the pal gene is represented by a nucleic acid sequence having at least 80%, preferably at least 90%, more preferably at least 95% nucleic acid sequence identity to SEQ ID NO: 7.

In one embodiment, the pal gene is represented by a nucleic acid sequence consisting of SEQ ID NO: 7.

In certain embodiments, the Pal protein refers to a preprotein with the UniProtKB access number P0A912. In some embodiments, the Pal preprotein is represented by an amino acid sequence having at least 75% amino acid sequence identity to SEQ ID NO: 8.

In some embodiments, the Pal preprotein is represented by an amino acid sequence having at least 80%, preferably at least 90%, more preferably at least 95% amino acid sequence identity to SEQ ID NO: 8.

In one embodiment, the Pal preprotein is represented by an amino acid sequence consisting of SEQ ID NO: 8.

In one embodiment, the mutation in the gene pal is a mutation disrupting the binding of Pal to the periplasmic peptidoglycan. As used herein, the expression “disrupting the binding of Pal to the periplasmic peptidoglycan” refers to a level of binding of Pal to the periplasmic peptidoglycan reaching at most about 75% of the level of binding observed in bacterium with unaltered envelop integrity.

In practice, the means to evaluate the binding of Pal to the periplasmic peptidoglycans are known to the skilled artisan and include the same techniques as to evaluate the binding of OmpA or Lpp to the periplasmic peptidoglycan.

Similarly to OmpA and Lpp, Pal is naturally synthesized as a 173 aa-long preprotein, which is subsequently cleaved during its addressing to the periplasm. This cleavage releases a 21 aa-long N-terminal signal peptide and a 152 aa-long mature protein. In practice, the position of the mutations in the pal gene may be defined with respect to the corresponding codon encoding the amino acid at a given position, taking as a reference the mature Pal protein of amino acid sequence SEQ ID NO: 9.

In one embodiment, the mutation in the pal gene is selected in the group comprising, or consisting of, a complete deletion of the pal gene and the substitution of the codon encoding arginine (R) at position 104 with a codon encoding a neutrally or negatively charged amino acid, preferably glutamic acid (E); wherein said position is defined with respect to the amino acid sequence SEQ ID NO: 9.

In one embodiment, the genetically modified gram-negative bacterium of the invention, in particular E. coli bacterium, comprises at least one mutation in the ybiS (also referred to as ldtB), ycfS (also referred to as ldtC) and/or erfK (also referred to as ldtA) gene(s).

The ybiS gene, the ycfS gene and the erfK gene each encodes an enzyme YbiS (also referred to as LdtB), YcfS (also referred to as LdtC) and ErfK (also referred to as LdtA), respectively, catalyzing the covalent binding of the mature Lpp protein via its C-terminal lysine to the periplasmic peptidoglycan. It is understood that the ybiS gene, the ycfS gene and the erfK gene encode proteins involved in the envelope integrity of a gram-negative bacterium according to the invention. More precisely, the ybiS gene, the ycfS gene and the erfK gene encode proteins involved in the attachment of the Lpp outer membrane protein to the periplasmic peptidoglycan.

In certain embodiments, the ybiS gene refers to a nucleic acid with the EcoCyc accession number G6422. In some embodiments, the ybiS gene is represented by a nucleic acid sequence having at least 75% nucleic acid sequence identity to SEQ ID NO: 10. In some embodiments, the ybiS gene is represented by a nucleic acid sequence having at least 80%, preferably at least 90%, more preferably at least 95% nucleic acid sequence identity to SEQ ID NO: 10. In one embodiment, the ybiS gene is represented by a nucleic acid sequence consisting of SEQ ID NO: 10.

In certain embodiments, the YbiS protein refers to a preprotein with the UniProtKB access number P0AAX8. In some embodiments, the YbiS preprotein is represented by an amino acid sequence having at least 75% amino acid sequence identity to SEQ ID NO: 11. In some embodiments, the YbiS preprotein is represented by an amino acid sequence having at least 80%, preferably at least 90%, more preferably at least 95% amino acid sequence identity to SEQ ID NO: 11. In one embodiment, the YbiS preprotein is represented by an amino acid sequence consisting of SEQ ID NO: 11.

In some embodiments, the homologue, in particular the functional homologue, of the ybiS gene is the ldtD, ldtE or ldtF gene. In certain embodiments, the homologue, in particular the functional homologue, of the YbiS protein is the LdtD, LdtE or LdtF protein. In some embodiments, the ldtD gene refers to a nucleic acid with the EcoCyc accession number EG11253. In certain embodiments, the ldtE gene refers to a nucleic acid with the EcoCyc accession number G6904. In some embodiments, the ldtF gene refers to a nucleic acid with the EcoCyc accession number G6108.

In certain embodiments, the ycfS gene refers to a nucleic acid with the EcoCyc accession number G6571. In some embodiments, the ycfS gene is represented by a nucleic acid sequence having at least 75% nucleic acid sequence identity to SEQ ID NO: 12. In some embodiments, the ycfS gene is represented by a nucleic acid sequence having at least 80%, preferably at least 90%, more preferably at least 95% nucleic acid sequence identity to SEQ ID NO: 12. In one embodiment, the ycfS gene is represented by a nucleic acid sequence consisting of SEQ ID NO: 12.

In certain embodiments, the YcfS protein refers to a preprotein with the UniProtKB access number P75954. In some embodiments, the YcfS preprotein is represented by an amino acid sequence having at least 75% amino acid sequence identity to SEQ ID NO: 13. In some embodiments, the YcfS preprotein is represented by an amino acid sequence having at least 80%, preferably at least 90%, more preferably at least 95% amino acid sequence identity to SEQ ID NO: 13. In one embodiment, the YcfS preprotein is represented by an amino acid sequence consisting of SEQ ID NO: 13.

In certain embodiments, the erfK gene refers to a nucleic acid with the EcoCyc accession number G7073. In some embodiments, the erfK gene is represented by a nucleic acid sequence having at least 75% nucleic acid sequence identity to SEQ ID NO: 14. In some embodiments, the erfK gene is represented by a nucleic acid sequence having at least 80%, preferably at least 90%, more preferably at least 95% nucleic acid sequence identity to SEQ ID NO: 14. In one embodiment, the erfK gene is represented by a nucleic acid sequence consisting of SEQ ID NO: 14.

In certain embodiments, the ErfK protein refers to a preprotein with the UniProtKB access number P39176. In some embodiments, the ErfK preprotein is represented by an amino acid sequence having at least 75% amino acid sequence identity to SEQ ID NO: 15. In some embodiments, the ErfK preprotein is represented by an amino acid sequence having at least 80%, preferably at least 90%, more preferably at least 95% amino acid sequence identity to SEQ ID NO: 15. In one embodiment, the ErfK preprotein is represented by an amino acid sequence consisting of SEQ ID NO: 15.

In certain embodiments, the mutated ybiS gene, ycfS gene and/or erfK gene, and/or a homologue thereof, consist in a deletion of said ybiS, ycfS and/or erfK genes, and/or a homologue thereof, respectively.

In one embodiment, the genetically modified gram-negative bacterium of the invention, in particular E. coli bacterium, comprises mutated ybiS and ycfS genes, and/or a homologue thereof. In one embodiment, the genetically modified gram-negative bacterium of the invention, in particular E. coli bacterium, comprises mutated ybiS and erfK genes, and/or a homologue thereof. In one embodiment, the genetically modified gram-negative bacterium of the invention, in particular E. coli bacterium, comprises mutated ycfS and erfK genes, and/or a homologue thereof. In one embodiment, the genetically modified gram-negative bacterium of the invention, in particular E. coli bacterium, comprises mutated ybiS, ycfS and erfK genes, and/or a homologue thereof.

It is understood herein that a mutation in any one of the ybiS, ycfS and/or erfK genes, and/or a homologue thereof, may result in the absence of a functional covalent binding of the mature Lpp polypeptide to the periplasmic peptidoglycan.

Techniques to measure the binding of Lpp to peptidoglycan are described hereinabove.

In one embodiment, the mutation in the ybiS gene, ycfS gene and/or erfK gene, and/or a homologue thereof, is selected in the group comprising, or consisting of, a complete deletion of ybiS, ycfS and/or erfK, and/or a homologue thereof, a complete deletion of ybiS and erfK, and/or a homologue thereof, and a complete deletion of ybiS, and ycfS, and/or a homologue thereof.

In one embodiment, the mutation in the ybiS gene, ycfS gene and/or erfK gene, and/or a homologue thereof, comprises, or consists of, a mutation impairing the catalytic site of the enzyme encoded by these genes.

In one embodiment, the genetically modified gram-negative bacterium of the invention, in particular E. coli bacterium, comprises at least the following mutations:
at least one mutation in the ompA gene, and/or a homologue thereof, comprising a substitution of the codon encoding arginine (R) at position 256 with a codon encoding a negatively or neutrally charged amino acid, preferably glutamic acid (E) or alanine (A); a substitution of the codon encoding aspartic acid (D) at position 241 with a codon encoding a neutrally or positively charged amino acid, preferably an asparagine (N); a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3; and,
at least one mutation in the lpp gene, and/or a homologue thereof, comprising a deletion of the codon encoding lysine (K) at position 58; a substitution of the codon encoding arginine (R) at position 57 with a codon encoding another amino acid, preferably a neutrally charged amino acid, more preferably leucine (L); a substitution of the codon encoding lysine (K) at position 58 with a codon encoding an arginine (R); or a complete deletion of the lpp gene; wherein said positions are defined with respect to the amino acid sequence SEQ ID NO: 6.

In one embodiment, the genetically modified gram-negative bacterium of the invention, in particular E. coli bacterium, comprises at least the following mutations:
at least one mutation in the lpp gene, and/or a homologue thereof, comprising a deletion of the codon encoding lysine (K) at position 58; a substitution of the codon encoding arginine (R) at position 57 with a codon encoding another amino acid, preferably a negatively or neutrally charged amino acid, more preferably leucine (L); a substitution of the codon encoding lysine (K) at position 58 with a codon encoding an arginine (R); or a complete deletion of the lpp gene; wherein said positions are defined with respect to the amino acid sequence SEQ ID NO: 6; and,
at least one mutation in the pal gene comprising a complete deletion of the pal gene or the substitution of the codon encoding arginine (R) at position 104 with a codon encoding a neutrally or negatively charged amino acid, preferably glutamic acid (E); wherein said position is defined with respect to the amino acid sequence SEQ ID NO: 9.

In one embodiment, the genetically modified gram-negative bacterium of the invention, in particular E. coli bacterium, or a variant thereof, comprises at least the following mutations:
at least one mutation in the ompA gene, and/or a homologue thereof, comprising a substitution of the codon encoding arginine (R) at position 256 with a codon encoding a negatively or neutrally charged amino acid, preferably glutamic acid (E) or alanine (A); a substitution of the codon encoding aspartic acid (D) at position 241 with a codon encoding a neutrally or positively charged amino acid, preferably asparagine (N); a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3; and,
a mutation in the pal gene, comprising a complete deletion of the pal gene or the substitution of the codon encoding arginine (R) at position 104 with a codon encoding a neutrally or negatively charged amino acid, preferably glutamic acid (E); wherein said position is defined with respect to the amino acid sequence SEQ ID NO: 9.

In one embodiment, the genetically modified gram-negative bacterium of the invention, in particular E. coli bacterium, comprises at least the following mutations:
at least one mutation in the ompA gene, and/or a homologue thereof, comprising a substitution of the codon encoding arginine (R) at position 256 with a codon encoding a negatively or neutrally charged amino acid, preferably glutamic acid (E) or alanine (A); a substitution of the codon encoding aspartic acid (D) at position 241 with a codon encoding a neutrally or positively charged amino acid, preferably an asparagine (N); a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3; and,
at least one mutation in each of at least one of, preferably at least two of, more preferably three of, the group of genes comprising the ybiS, ycfS and erfK genes, and/or a homologue thereof; preferably wherein said mutation(s) is/are selected in the group comprising, or consisting of, a complete deletion of said ybiS, ycfS and erfK genes, and/or a homologue thereof.

In one embodiment, the genetically modified gram-negative bacterium of the invention, in particular E. coli bacterium, or a variant thereof, comprises at least the following mutations:
at least one mutation in the ompA gene, and/or a homologue thereof, comprising a substitution of the codon encoding arginine (R) at position 256 with a codon encoding a neutrally or negatively charged amino acid, preferably glutamic acid (E) or alanine (A); a substitution of the codon encoding aspartic acid (D) at position 241 with a codon encoding a neutrally or positively charged amino acid, preferably asparagine (N); a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3;
at least one mutation in the lpp gene, and/or a homologue thereof, comprising a deletion of the codon encoding lysine (K) at position 58; a substitution of the codon encoding arginine (R) at position 57 with a codon encoding another amino acid, preferably a neutrally charged amino acid, more preferably leucine (L); a substitution of the codon encoding lysine (K) at position 58 with a codon encoding an arginine (R); or a complete deletion of the lpp gene; wherein said positions are defined with respect to the amino acid sequence SEQ ID NO: 6; and,
at least one mutation in each of at least one of, preferably at least two of, more preferably at least three of, the group of genes comprising the ybiS gene, the ycfS gene and the erfK gene, and/or a homologue thereof; preferably wherein said mutation(s) is/are selected in the group comprising, or consisting of, a complete deletion of said ybiS, ycfS and erfK genes, and/or a homologue thereof.

In one embodiment, the genetically modified gram-negative bacterium of the invention, in particular E. coli bacterium, comprises at least the following mutations:
a complete deletion of the lpp gene and/or a homologue thereof; and
a mutation in the ompA gene consisting of the deletion of the C-terminal part of the OmpA protein starting at or before the codon encoding aspartic acid (D) at position 241 or arginine (R) at position 256, wherein said position is defined with respect to the amino acid sequence SEQ ID NO: 3.

In one embodiment, the genetically modified gram-negative bacterium of the invention, in particular E. coli bacterium, comprises at least the following mutations:
a complete deletion of the lpp gene and/or a homologue thereof; and
a complete deletion of the ompA gene and/or a homologue thereof.

In one embodiment, the genetically modified gram-negative bacterium of the invention, in particular E. coli bacterium, does not comprise at least the following mutations:
a complete deletion of the lpp gene and/or a homologue thereof; and
a complete deletion of the ompA gene and/or a homologue thereof.

In one embodiment, the genetically modified gram-negative bacterium of the invention, in particular E. coli bacterium, comprises at least the following mutations:
a mutation in the ompA gene, and/or a homologue thereof, consisting of the substitution of the codon encoding arginine (R) at position 256 with a codon encoding glutamic acid (E), wherein said position is defined with respect to the amino acid sequence SEQ ID NO: 3; and
a complete deletion of the pal gene.

In one embodiment, the genetically modified gram-negative bacterium of the invention, in particular E. coli bacterium, comprises at least the following mutations:
a mutation in the lpp gene, and/or a homologue thereof, consisting of the deletion of the codon encoding lysine (K) at position 58, wherein said position is defined with respect to the amino acid sequence SEQ ID NO: 6; and
a complete deletion of the pal gene.

In one embodiment, the genetically modified gram-negative bacterium of the invention, in particular E. coli bacterium, comprises at least the following mutations:
a mutation in the ompA gene consisting of the substitution of the codon encoding arginine (R) at position 256 with a codon encoding a glutamic acid (E), wherein said position is defined with respect to the amino acid sequence SEQ ID NO: 3, and
a mutation in the lpp gene consisting of the deletion of the codon encoding lysine (K) at position 58, wherein said position is defined with respect to the amino acid sequence SEQ ID NO: 6.

In one embodiment, the genetically modified gram-negative bacterium of the invention, in particular E. coli bacterium, comprises at least the following mutations:
a deletion of the C-terminal part of the OmpA protein starting at or before the codon encoding aspartic acid (D) at position 241 or arginine (R) at position 256, wherein said position is defined with respect to the amino acid sequence SEQ ID NO: 3, and
a mutation in the lpp gene consisting of the deletion of the codon encoding lysine (K) at position 58, wherein said position is defined with respect to the amino acid sequence SEQ ID NO: 6.

In one embodiment, the genetically modified gram-negative bacterium of the invention, in particular E. coli bacterium, comprises at least the following mutations:
a mutation in the lpp gene, and/or a homologue thereof, consisting of the substitution of the codon encoding arginine (R) at position 57 with a codon encoding a leucine (L), wherein said position is defined with respect to the amino acid sequence SEQ ID NO: 6;
a complete deletion of the ybiS gene, and/or a homologue thereof; and
a complete deletion of the ycfS gene, and/or a homologue thereof.

In one embodiment, the genetically modified gram-negative bacterium of the invention, in particular E. coli bacterium, comprises at least the following mutations:
a mutation in the ompA gene, and/or a homologue thereof, consisting of the substitution of the codon encoding arginine (R) at position 256 with a codon encoding a glutamic acid (E), wherein said position is defined with respect to the amino acid sequence SEQ ID NO: 3,
a mutation in the lpp gene, and/or a homologue thereof, consisting of the substitution of the codon encoding arginine (R) at position 57 with a codon encoding a leucine (L), wherein said position is defined with respect to the amino acid sequence SEQ ID NO: 6;
a complete deletion of the ybiS gene, and/or a homologue thereof; and
a complete deletion of the erfK gene, and/or a homologue thereof.

In one embodiment, the genetically modified gram-negative bacterium of the invention, in particular E. coli bacterium, comprises at least the following mutations:
a mutation in the ompA gene, consisting of the substitution of the codon encoding arginine (R) at position 256 with a codon encoding a glutamic acid (E), wherein said position is defined with respect to the amino acid sequence SEQ ID NO: 3,
a mutation in the lpp gene, consisting of the substitution of the codon encoding arginine (R) at position 57 with a codon encoding a leucine (L), wherein said position is defined with respect to the amino acid sequence SEQ ID NO: 6;
a complete deletion of the ybiS gene; and
a complete deletion of the ycfS gene.

In one embodiment, the genetically modified gram-negative bacterium of the invention, in particular E. coli bacterium, comprises at least the following mutations:
a mutation in the ompA gene, and/or a homologue thereof, consisting of the substitution of the codon encoding arginine (R) at position 256 with a codon encoding a glutamic acid (E), wherein said position is defined with respect to the amino acid sequence SEQ ID NO: 3,
a mutation in the lpp gene, and/or a homologue thereof, consisting of the substitution of the codon encoding arginine (R) at position 57 with a codon encoding a leucine (L), wherein said position is defined with respect to the amino acid sequence SEQ ID NO: 6;
a complete deletion of the ycfS gene, and/or a homologue thereof; and
a complete deletion of the erfK gene, and/or a homologue thereof.

In one embodiment, the genetically modified gram-negative bacterium of the invention, in particular E. coli bacterium, comprises at least the following mutations:
a mutation in the ompA gene consisting of the substitution of the codon encoding arginine (R) at position 256 with a codon encoding a glutamic acid (E), wherein said position is defined with respect to the amino acid sequence SEQ ID NO: 3,
a complete deletion of the ybiS gene,
a complete deletion of the ycfS gene, and,
a complete deletion of the erfK gene.

In one embodiment, the genetically modified gram-negative bacterium of the invention, in particular E. coli bacterium, comprises at least the following mutations:
a mutation in the ompA gene consisting of the substitution of the codon encoding aspartic acid (D) at position 241 with a codon encoding an asparagine (N), wherein said position is defined with respect to the amino acid sequence SEQ ID NO: 3; and
a complete deletion of the lpp gene.

In one embodiment, the genetically modified gram-negative bacterium of the invention, in particular E. coli bacterium, comprises at least the following mutations:
a mutation in the ompA gene, and/or a homologue thereof, consisting of the substitution of the codon encoding arginine (R) at position 256 with a codon encoding a glutamic acid (E), wherein said position is defined with respect to the amino acid sequence SEQ ID NO: 3; and
a mutation in the lpp gene, and/or a homologue thereof, consisting of the deletion of the codon encoding lysine (K) at position 58, wherein said position is defined with respect to the amino acid sequence SEQ ID NO: 6.

In certain embodiments, the bacterium according to the instant invention is selected in a group of bacteria having (i) a mutation in the ompA gene consisting of the substitution of the codon encoding arginine (R) at position 256 with a codon encoding a glutamic acid (E) wherein said position is defined with respect to the amino acid sequence SEQ ID NO: 3, and a mutation in the lpp gene consisting of the deletion of the codon encoding lysine (K) at position 58, wherein said position is defined with respect to the amino acid sequence SEQ ID NO: 6; (ii) a mutation in the ompA gene consisting of the substitution of the codon encoding arginine (R) at position 256 with a codon encoding a glutamic acid (E) wherein said position is defined with respect to the amino acid sequence SEQ ID NO: 3, and complete deletion of the pal gene; or (iii) a mutation in the lpp gene consisting of the deletion of the codon encoding lysine (K) at position 58, wherein said position is defined with respect to the amino acid sequence SEQ ID NO: 6, and a complete deletion of the pal gene.

In one embodiment, the mutations in each of at the least two mutated genes encoding a protein involved in the envelope integrity, are genomic mutations.

As used herein, the expression “genomic mutation” refers to a mutation in a nucleic acid sequence from the bacterial chromosome. In such embodiment, the mutation is stable and transmitted to the progeny of the bacterial cell comprising said mutation.

In certain embodiments, the bacterium according to the invention further comprises at least one extra-genomic nucleic acid molecule.

In certain embodiments, the bacterium according to the invention further comprises at least one extra-genomic nucleic acid molecule, preferably encoding at least one polypeptide.

In one embodiment, the at least one extra-genomic nucleic acid molecule is selected from the group comprising, or consisting of, a plasmid, a cosmid or a bacterial artificial chromosome (BAC).

In practice, the extra-genomic nucleic acid molecule may be in the form of a plasmid, in particular resulting from the cloning of a nucleic acid molecule of interest into a nucleic acid vector. In some embodiments, non-limitative suitable nucleic acid vectors are pBluescript vectors, pET vectors, pETduet vectors, pGBM vectors, pBAD vectors, pUC vectors. In one embodiment, the plasmid is a low copy plasmid. In one embodiment, the plasmid is a high copy plasmid.

In practice, the extra-genomic nucleic acid molecule may comprise a nucleic acid molecule of therapeutic interest, such as, e.g., for vaccination or gene therapy.

In practice, a nucleic acid molecule of therapeutic interest may be e.g. an antisense oligonucleotide, an aptamer, or may encode a micro RNA, such as e.g., a short interfering RNA (siRNA).

In some embodiments, when the synthesis of a polypeptide encoded by a nucleic acid molecule is envisioned, the nucleic acid vector may also comprise a promoter that is inducible, in particular the promoter of the lacZ gene, the promoter of the trp gene or the promoter of the β-lactamase encoding gene. In practice, the nucleic acid vector may also comprise a nucleic acid encoding the resistance to an antibiotic, in particular, ampicillin, kanamycin, chloramphenicol, tetracycline, spectinomycin or streptomycin.

In certain embodiments, a polypeptide encoded by a nucleic acid molecule is of therapeutic interest. In some embodiments, the polypeptide is a cytoplasmic polypeptide.

In some embodiments, the polypeptide is a non-secreted polypeptide.

As used herein, “non-secreted polypeptide” refers to a polypeptide that is synthesized within the bacterium cytoplasm and that does not embed into or cross the bacterial membrane, including the cytoplasmic membrane and the outer membrane of a gram-negative bacterium, in particular E. coli. In other words, “non-secreted polypeptide” refers to a polypeptide that is not embedded into the cytoplasmic membrane, not targeted into the periplasm, not embedded into the outer membrane, or not targeted into the culture medium. Within the scope of the instant invention, the terms “cytoplasmic membrane” and “inner membrane” are meant to be equivalent.

In one embodiment, the therapeutic polypeptide is selected in a group comprising a protein with enzymatic or regulatory activity, a protein with special targeting activity, a protein with vaccine properties and a protein with diagnostic properties.

In practice, a therapeutic polypeptide encoded by a nucleic acid molecule of interest may be e.g. a growth factor, an antibody, a hormone, a cytokine, an enzyme, a plasmatic factor, and the likes. Illustratively, therapeutic polypeptides for use according to the instant invention may be implemented in methods for the treatment and/or the prevention of disorders or diseases, such as e.g. an anemia, an autoimmune disease, a cancer, a diabetes, a hemophilia, an infectious disease and a neurodegenerative disease.

Within the scope of the invention, the use and the methods of the invention may be performed in vivo or in vitro.

It is understood that the bacteria according to the invention may be implemented for the industrial production and purification of extra-genomic nucleic acids, e.g. plasmids, and/or polypeptides encoded by said nucleic acids.

The invention relates to the use of a genetically modified E. coli bacterium comprising at least one extra-genomic nucleic acid molecule and comprising at least one mutated gene encoding a protein involved in the envelope integrity, said bacterium having an altered envelop integrity as compared to a bacterium with unaltered envelop integrity, wherein the at least one mutated gene is ompA, and/or a homologue thereof, or a gene involved in Lpp functionality, for the production and the purification of the at least one extra-genomic nucleic acid molecule.

As used herein, “at least one” encompasses 1, 2, 3, 4, 5, 6, 7, 8, 9, 10 or more.

Another aspect of the invention relates to the use of a genetically modified gram-negative bacterium according to the instant invention, in particular E. coli, for the production and the purification of at least one extra-genomic nucleic acid molecule.

In certain embodiments, the at least one extra-genomic nucleic acid molecule is selected in the group comprising or consisting of a plasmid, a cosmid and a bacterial artificial chromosome (BAC).

A further aspect of the invention pertains to the use of genetically modified E. coli bacterium comprising at least one extra-genomic nucleic acid molecule encoding at least one polypeptide and comprising at least one mutated gene encoding a protein involved in the envelope integrity, said bacterium having an altered envelop integrity as compared to a bacterium with unaltered envelop integrity, wherein the at least one mutated gene is ompA, and/or a homologue thereof, or a gene involved in Lpp functionality, for the production and the purification of the at least one polypeptide, preferably encoded by the at least one extra-genomic nucleic acid molecule.

In some embodiments, the bacterium is oversensitive to bacterial lysis as compared to a bacterium with unaltered envelop integrity.

A still other aspect of the invention pertains to the use of a genetically modified gram-negative bacterium according to the invention, in particular E. coli, for the production and the purification of at least one polypeptide, preferably encoded by at least one extra-genomic nucleic acid molecule.

In certain embodiments, the polypeptide is encoded by a genomic nucleic acid.

In some embodiments, the at least one polypeptide is at least one cytoplasmic polypeptide.

In certain embodiments, the at least one polypeptide is at least one non-secreted polypeptide.

In certain embodiments, the at least a mutated ompA gene consists of a substitution of the codon encoding arginine (R) at position 256 with a codon encoding a neutrally or negatively charged amino acid, preferably glutamic acid (E) or alanine (A); and/or a substitution of the codon encoding aspartic acid (D) at position 241 with a codon encoding a neutrally or positively charged amino acid, preferably asparagine (N); and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

In some embodiments, the mutated gene involved in Lpp functionality consists of a mutation in the lpp gene consisting of the deletion of the codon encoding lysine (K) at position 58, wherein said position being defined with respect to the amino acid sequence SEQ ID NO: 6, or the complete deletion of ybiS gene, and/or the complete deletion of the ycfS gene and/or the complete deletion of the erfK gene.

In some embodiments, the bacterium comprises at least two mutated genes encoding proteins involved in the envelope integrity. In certain embodiments, the bacterium comprises at least two mutated genes encoding proteins involved in the envelope integrity, wherein at least one mutated gene is ompA, and/or a homologue thereof, and at least one mutated gene is a gene involved in Lpp functionality.

In one embodiment, the genetically modified gram-negative bacterium of the invention, in particular E. coli bacterium, does not comprise simultaneously a complete deletion of the ompA gene; and a complete deletion of the lpp gene.

In certain embodiments, the bacterium is as defined in the instant invention.

The invention also relates to a method for the production of at least one extra-genomic nucleic acid molecule comprising the steps of:
a) culturing genetically modified gram-negative bacteria according to the instant invention, in particular E. coli, comprising at least one extra-genomic nucleic acid molecule, so as to amplify the at least extra-genomic nucleic acid molecule; and
b) lysing the bacteria obtained at step a), preferably by chemical lysis so as to obtain a lysis mixture.

The invention also relates to a method for the production and the purification of at least one extra-genomic nucleic acid molecule comprising the steps of:
a) culturing genetically modified gram-negative bacteria according to the instant invention, in particular E. coli, comprising at least one extra-genomic nucleic acid molecule, so as to amplify the at least extra-genomic nucleic acid molecule;
b) lysing the bacteria obtained at step a), preferably by chemical lysis so as to obtain a lysis mixture; and,
c) purifying said extra-genomic nucleic acid molecule from the lysis mixture obtained at step b).

The invention also relates to a method for the production and the purification of at least one extra-genomic nucleic acid molecule comprising the steps of:
a) culturing genetically modified E. coli bacteria comprising at least one mutated gene encoding a protein involved in the envelope integrity, said bacteria having an altered envelop integrity and being oversensitive to bacterial lysis as compared to a bacterium with unaltered envelop integrity, wherein the at least one mutated gene is ompA, and/or a homologue thereof, or a gene involved in Lpp functionality, said bacteria comprising at least one extra-genomic nucleic acid molecule, so as to amplify the at least extra-genomic nucleic acid molecule;
b) lysing the bacteria obtained at step a), preferably by chemical lysis, so as to obtain a lysis mixture; and,
c) purifying said amplified extra-genomic nucleic acid molecule from the lysis mixture obtained at step b).

In certain embodiments, the at least one extra-genomic nucleic acid molecule is selected in the group comprising or consisting of a plasmid, a cosmid and a bacterial artificial chromosome (BAC).

In another aspect, the invention relates to a method for the production of at least one polypeptide, preferably encoded by an extra-genomic nucleic acid molecule, comprising the steps of:
a) culturing genetically modified gram-negative bacteria according to the instant invention, in particular E. coli, preferably comprising at least one extra-genomic nucleic acid molecule encoding the at least one polypeptide, so as to synthesize the at least one polypeptide; and
b) lysing the bacteria obtained at step a), so as to obtain a lysis mixture.

In another aspect, the invention relates to a method for the production and the purification of at least one polypeptide, preferably encoded by an extra-genomic nucleic acid molecule, comprising the steps of:
a) culturing genetically modified gram-negative bacteria according to the instant invention, in particular E. coli, preferably comprising at least one extra-genomic nucleic acid molecule encoding the at least one polypeptide, so as to synthesize the at least one polypeptide;
b) lysing the bacteria obtained at step a), so as to obtain a lysis mixture; and,
c) purifying said at least one polypeptide from the lysis mixture obtained at step b).

One further aspect of the invention relates to a method for the production and the purification of at least one polypeptide, preferably encoded by an extra-genomic nucleic acid molecule, comprising the steps of:
a) culturing genetically modified E. coli bacteria comprising at least one mutated gene encoding a protein involved in the envelope integrity, said bacteria having an altered envelop integrity and being oversensitive to bacterial lysis as compared to a bacterium with unaltered envelop integrity, wherein the at least one mutated gene is ompA, and/or a homologue thereof, or a gene involved in Lpp functionality, said bacteria preferably comprising at least one extra-genomic nucleic acid molecule encoding the at least one polypeptide, so as to synthesize the at least one polypeptide;
b) lysing the bacteria obtained at step a), so as to obtain a lysis mixture; and,
c) purifying said at least one polypeptide from a lysis mixture obtained at step b).

In certain embodiments, the polypeptide is encoded by a genomic nucleic acid.

In some embodiments, the at least one polypeptide is one cytoplasmic polypeptide.

In certain embodiments the at least one polypeptide is one non-secreted polypeptide.

In some embodiments, the bacterium from the above methods comprises at least two mutated genes encoding proteins involved in the envelope integrity. In certain embodiments, the bacterium from the above methods comprises at least two mutated genes encoding proteins involved in the envelope integrity, wherein at least one mutated gene is ompA and at least one mutated gene is a gene involved in Lpp functionality.

In one embodiment, the genetically modified gram-negative bacterium of the invention, in particular E. coli bacterium, does not comprise simultaneously a complete deletion of the ompA gene; and a complete deletion of the lpp gene.

In some embodiments, the methods of the instant invention may further comprise, prior to step a), a step a0) comprising, or consisting of, transforming a genetically modified gram-negative bacterium of the invention, in particular E. coli, with said at least one extra-genomic nucleic acid molecule.

In one embodiment, the polypeptide produced by the method of the invention is not secreted by the genetically modified gram-negative bacterium of the invention, in particular E. coli. Therefore, said polypeptide requires its release from the producing bacterium.

The term “transforming” is used herein to refer to the introduction of an extra-genomic nucleic acid molecule in the cytoplasm of a bacterium, in particular E. coli. The bacterium, in particular E. coli, the cytoplasm of which contains at least one extra-genomic nucleic acid molecule after the step of transformation, are qualified as “transformed bacterium”. The transformation of bacteria requires typically that said cells have been beforehand treated to become “competent” for the extra-genomic nucleic acid molecule to enter the cytoplasm upon transformation. As used herein, the term “competent” refers to a bacterium that has an increased ability to uptake an extra genomic nucleic acid molecule into the its cytoplasm. The skilled artisan is familiar with techniques for preparing competent bacteria. Illustratively, for the steps of preparation of competent bacteria, transformation, selection of transformed bacteria one may refer to the manufacturer's instructions, when commercial kits or materials are used, and/or refer to, e.g., the protocols described by J. Sambrook and D. Russell, Molecular Cloning: A Laboratory Manual, 3^{rd} ed., Cold Spring Harbor Laboratory Press, Cold Spring Harbor, N. Y. (2001).

In some embodiments, competent bacteria may be chemically competent cells, in particular calcium chloride treated bacteria. In some alternative embodiments, competent bacteria may be electrocompetent bacteria. In practice, chemically competent or electrocompetent bacteria may be purchased from THERMOFISHER®, SIGMA-ALDRICH® or NEB®. For E. coli bacteria, a non-limitative list of commercial chemically competent bacteria encompasses BL21(DE3), DH10B, DH5α, Machi, TOP10, INV110, SIG10. A non-limitative list of commercial E. coli electrocompetent bacteria encompasses Mega DH10B T1R, ElectroMAX DH5α, One shot TOP10, SIG10 MAX.

In one aspect, the invention relates to a genetically modified gram-negative bacterium according to the invention, in particular E. coli, in a competent form. In other words, the invention relates to a competent genetically modified gram-negative bacterium according to the invention, in particular E. coli. The competent genetically modified gram-negative bacterium may be chemically competent or electrocompetent.

It is understood that the bacteria according to the invention are cultured in an appropriate culture medium, so as to amplify the initial population of bacteria, in order to achieve the amplification of the extra-genomic nucleic acid molecule and/or to achieve significant synthesis of the polypeptide encoded by said nucleic acid molecule, in particular, a cytoplasmic polypeptide.

The skilled artisan is familiar with techniques for culturing bacteria, in particular E. coli. Briefly and for illustrative purposes, a suitable culture medium is inoculated with bacteria and incubated, at a constant temperature (from about 20° C. to about 40° C.), optimally in the case of E. coli a temperature of about 37° C., and under agitation, until the desired density of cell has been obtained. The cell density may be evaluated by measuring the optical density of the culture or by counting cell using a microscope. The culture medium is typically complemented with antibiotics matching the antibiotic resistance conferred by the presence of the at least one extra-genomic nucleic acid molecule of interest to maintain a selective pressure on bacteria. In practice, non-limitative examples of suitable culture media for bacterial growth encompass LB broth, Terrific broth and M9 minimal medium. Commercially available culture media may be purchased from e.g. SIGMA-ALDRICH®, THERMOFISHER®, to name a few companies. In the methods of the invention aiming at the production of a polypeptide, the culture medium may be complemented with an inducing molecule triggering the expression under the control of an inducible promoter of the nucleic acid sequence encoding said polypeptide. Illustratively, Isopropyl β-D-1-thiogalactopyranoside may be used to induce expression of a nucleic acid sequence under the control of the lac operator.

In certain embodiments, the polypeptide may be fused with a tag, for the ease of purification. Non-limiting examples of tags suitable for the invention may be selected in a group comprising a FLAG-tag, GST-tag, Halo-Tag, His-tag, MBP-tag, Snap-Tag, SUMO-tag and a combination thereof.

In practice, and for industrial purposes, the culture of the bacteria according to the invention may be performed in a suitable fermenter (bioreactor), such as e.g. a 5 L, 50 L, 100 L, 500 L or 1,000 L fermenter. The culture in the fermenter may be performed in batch or fed batch conditions.

As used herein, the expression “batch fermentation” is meant to refer to a fermentation achieved by loading substrates and bacteria into the fermenter batchwise. As used herein, the expression “fed-batch fermentation” refers to a fermentation in which a high concentration of a given substrate is toxic to the bacterial culture: with the aim of keeping the substrate concentration below toxic levels, said substrate is gradually added (“fed”) at a slow rate as the substrate is consumed by the culture.

It is understood that upon amplification of the extra genomic nucleic acid molecule and/or the production of the polypeptide encoded by said nucleic acid molecule, the nucleic acid molecule and/or the polypeptide is/are extracted from the bacteria. Said extraction is performed by a step of lysing the bacteria. Within the scope of the instant invention, the lysis is achieved so as to recover as much of the nucleic acid molecule and/or the polypeptide encoded by said nucleic acid molecule as possible, while avoiding extraction from the bacteria of the bacterial chromosome, the natural protein content of the bacteria and/or bacterial debris.

As used herein the term “lysing”, “bacterial lysis” and “lysis” are used to refer to the partial or complete disruption of the bacterial envelope such that the content of the cytoplasm is released, at least in part, outside the bacterium. The skilled artisan is familiar with techniques for lysing bacteria. Such techniques include, but are not limited to, mechanical lysis techniques, such as for example technique using an high pressure homogenizer, bead mills and sonication; enzymatic lysis techniques, such as for example, using lysozyme and/or proteinase K; thermal lysis, such as for example techniques using freeze/taw cycles; and chemical lysis techniques, such as for example, osmotic shock, alkaline lysis and detergent lysis and combinations thereof.

In one embodiment, the lysis of the genetically modified gram-negative bacterium of the invention, in particular E. coli, is a chemical lysis, preferably an alkaline lysis.

As used herein, the expression “chemical lysis” is meant to refer to a lysis technique generally based upon the incubation of bacteria in solution(s) comprising particular solutes, such as ions and/or detergents, leading to the disruption of the bacterial membrane. As used herein, the expression “alkaline lysis” is meant to refer to a lysis method based on the incubation of bacteria in a solution comprising OH⁻ ions and sodium dodecyl sulphate (SDS).

In practice, the final concentration of OH— ions for the lysis step is from about 50 mM to about 500 mM, preferably from about 75 mM to about 250 mM. Within the scope of the instant invention, the expression “from about 50 mM to about 500 mM” encompasses 50 mM, 55 mM, 60 mM, 65 mM, 70 mM, 75 mM, 80 mM, 85 mM, 90 mM, 95 mM, 100 mM, 110 mM, 120 mM, 125 mM, 130 mM, 140 mM, 150 mM, 160 mM, 170 mM, 175 mM, 180 mM, 190 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM and 500 mM.

In practice, a source of OH— ions may be NaOH.

In practice, the final concentration of SDS for the lysis step is from about 0.1% to about 5%, preferably from about 0.2% to about 2%, more preferably from about 0.25% to about 0.75%. Within the scope of the instant invention, the expression “from about 0.1% to about 5%” encompasses 0.1%, 0.2%, 0.25%, 0.3%, 0.4%, 0.5%, 0.6%, 0.7%, 0.75%, 0.8%, 0.9%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, and 5%.

Without being bound by a theory, the OH⁻ ions may react with the bacterial membrane and break the fatty acid-glycerol ester bonds and subsequently permeabilize the bacterial membrane to the SDS, which in turn may solubilize the proteins and the membrane. In addition, NaOH may denature the cellular DNA, which becomes linearized and which strands are separated, while the circular plasmidic DNA remains topologically constrained. Illustratively, performing the steps of alkaline lysis may be achieved with commercial kits, such as for example the mini, midi and maxi prep kits from Qiagen® or the Macherey-Nagel® kit, according to the manufacturer's instructions. Alternatively, one may refer to the detailed protocols described in J. Sambrook and D. Russell, Molecular Cloning: A Laboratory Manual, 3^{rd} ed., Cold Spring Harbor Laboratory Press, Cold Spring Harbor, N. Y. (2001).

In one embodiment, the lysis step of the genetically modified gram-negative bacterium of the invention, in particular E. coli, comprises a step of subjecting said bacterium to an osmotic shock.

The expression “osmotic shock”, as used herein, corresponds to a sudden change (e.g., over a duration at or below about 5 minutes) in the osmotic concentration of the solution comprising the genetically modified gram-negative bacterium of the invention, in particular E. coli. As used herein, the expression “osmotic concentration” refers to the measure of the solute concentration expressed in number of osmoles of solute per liter (Osm/L) or per kilogram of solvent (Osm/kg).

In one embodiment, the amplitude of said osmotic shock corresponds to a decrease in the osmotic concentration of the solution comprising the genetically modified gram-negative bacterium of the invention, in particular E. coli, by a factor of, or below, about 0.9, preferably by a factor of, or below, about 0.8, 0.7 or 0.6, more preferably by a factor of, or below, about 0.5, 0.4 or 0.3, even more preferably by a factor of, or below, about 0.25, 0.2, 0.15 or 0.1, even further more preferably by a factor of, or below, about 0.095 or less. In one embodiment, the amplitude of said osmotic shock corresponds to a decrease in the osmotic concentration of the solution comprising the genetically modified gram-negative bacterium of the invention, in particular E. coli, by a factor of at least about 10%, preferably by a factor of at least about 15%, 20%, 25%, more preferably by a factor of at least about 30%, 40%, 50%, 60%, 70%, 80%, 90%. In some embodiments, the factor is 100%. Within the scope of the instant invention, a factor of 100% is intended to mean that the bacteria are suspended in a medium or buffer having an osmolarity of about 0 mOsm/L.

Illustratively, the LB culture medium has an osmolarity of 440 mOsm/L. When pure water is used for implementing an osmotic shock, the osmolarity drops from 440 mOsm/L to 0 mOsm/L. Therefore, the decrease of osmolarity is 100%.

In one embodiment, the step of subjecting the genetically modified gram-negative bacterium of the invention to an osmotic shock comprises the incubation in pure water for at least about 30 seconds, preferably for at least about 45 seconds, 60 seconds, 75 seconds, 100 seconds, 125 seconds, 150 seconds, 175 seconds, 200 seconds, 225 seconds, 250 seconds or 275 seconds, more preferably for at least about 300 seconds.

In one embodiment, the sensitivity of the genetically modified gram-negative bacterium of the invention, in particular E. coli, to an osmotic shock is increased. In said embodiment, the genetically modified gram-negative bacterium of the invention, in particular E. coli, is referred to as oversensitive to nucleic acid molecule or polypeptide extraction, preferably oversensitive to extra-genomic nucleic acid molecule or polypeptide extraction, as compared to a bacterium without unaltered envelop integrity.

The sensitivity of bacteria to an osmotic shock may be evaluated by quantifying the proportion of surviving bacteria (e.g. able to proliferate) upon an osmotic shock.

The survival of bacteria upon an osmotic shock may be evaluated by measuring the ratio [CFU/mL]ₒₛ/[CFU/mL]_{T0}, wherein [CFU/mL]ₒₛ represents the number of colony forming units (CFU) per mL after the osmotic shock and [CFU/mL]_{T0} represents the number of CFU/mL before the osmotic shock.

The number of CFU/mL may be measured according to the common knowledge in the art, in particular following 1:10 serial dilutions of a sample of bacteria in fresh medium, depositing a sample of said dilutions onto an agar-containing solid culture medium and counting the CFU in the suitable corresponding dilutions. Alternatively, the CFU/mL may be evaluated by measuring the optical density at about 600 nm.

In one embodiment, genetically modified gram-negative bacteria that are oversensitive to an osmotic shock have a ratio [CFU/mL]ₒₛ/[CFU/mL]_{T0} of from about 1:10¹ to about 1:10⁶, preferably of from about 1:10² to about 1:10⁵. Within the scope of the instant invention, the expression “from about 1:10¹ to about 1:10⁶” encompasses 1:10¹, 1:10², 1:10³, 1:10⁴, 1:10⁵ and 1:10⁶.

In certain embodiments, the ratio [CFU/mL]ₒₛ/[CFU/mL]_{T0} may be expressed in log (log₁₀). A difference of 2 logs corresponds to a ratio of 1:10², whereas a difference of 5 logs corresponds to a ratio of 1:10⁵.

In one embodiment, the amount per cell, or per mL of culture, of at least one extra-genomic nucleic acid molecule and/or polypeptide, released from the genetically modified gram-negative bacterium of the invention upon lysis, preferably chemical lysis, more preferably alkaline lysis, is increased as compared to the amount released from a bacterium with unaltered envelop integrity in comparable conditions. In said embodiment, the genetically modified gram-negative bacterium of the invention, in particular E. coli, is referred to as being oversensitive to nucleic acid molecule and/or polypeptide extraction, preferably oversensitive to extra-genomic nucleic acid molecule and/or polypeptide extraction.

In one embodiment, the yield may be evaluated by calculating a ratio [AM/cell]_{BA1}/[AM/cell]_{BR}, wherein [AM/cell]_{BA1} refers to the amount of nucleic acid molecules or polypeptide recovered from a bacterium according to the invention following the method disclosed herein and [AM/cell]_{BR} refers to the amount of nucleic acid molecules or polypeptide recovered from a reference bacterium, i.e. a bacterium with unaltered envelop integrity, following the same method.

In one embodiment, the amount per cell, or per mL of culture, of at least one extra-genomic nucleic acid molecule and/or polypeptide, released from the genetically modified gram-negative bacterium of the invention, upon lysis, preferably chemical lysis, more preferably alkaline lysis, increases by a factor of about, or at least about, 1.1, preferably by a factor of about, or at least about, 1.2, 1.3, 1.4, 1.5, 1.6, 1.7, 1.8 or 1.9, more preferably by a factor of about, or at least about, 1.9 or more, when compared to a reference bacterium, i.e. a bacterium with unaltered envelop integrity. In said embodiment, the genetically modified gram-negative bacterium of the invention, in particular E. coli, is referred as being oversensitive to nucleic acid and/or polypeptide extraction, preferably oversensitive to extra-genomic nucleic acid or polypeptide extraction.

In one embodiment, the ratio of the amount per cell, or per mL of culture, of at least one extra-genomic nucleic acid molecule and/or polypeptide released from the genetically modified gram-negative bacterium of the invention, in particular E. coli, over the amount of genomic DNA released from the genetically modified gram-negative bacterium of the invention upon lysis, preferably alkaline lysis, is from at least about 1:1 to at least 10:1, including 1:1, 2:1, 3:1; 4:1, 5:1, 6:1, 7:1, 8:1 9:1 and 10:1.

In one embodiment, the amount per cell, or per mL, of culture of at least one polypeptide released from the genetically modified gram-negative bacterium of the invention, in particular E. coli, upon lysis, preferably chemical lysis, more preferably alkaline lysis, increases by a factor of about, or at least about, 1.1, preferably by a factor of about, or at least about, 1.2, 1.3, 1.4, 1.5, 1.6, 1.7, 1.8 or 1.9, preferably by a factor of about, or at least about, 2 or more, when compared to a reference gram-negative bacterium, in particular E. coli, i.e. a bacterium with an unaltered envelop integrity.

In one embodiment, the amount per cell, or per mL of culture, of at least one polypeptide and/or at least one extra-genomic nucleic acid molecule, recited hereinabove correspond to amount after a step of purification.

In one embodiment, the amount per cell, or per mL of culture, of at least one extra-genomic nucleic acid molecule, recited hereinabove correspond to amount of super-coiled plasmid.

In one embodiment, the methods of the invention comprise after lysis, a step of purifying the at least one extra-genomic nucleic acid molecule released by the genetically modified gram-negative bacterium of the invention, in particular E. coli, which step corresponds to step c) of the methods disclosed herein.

In one embodiment, the methods of the invention comprise a step of purifying the at least one polypeptide released by the genetically modified gram-negative bacterium of the invention, in particular E. coli.

The skilled artisan is familiar with techniques to purify nucleic acids and/or proteins. Illustratively, for the steps of purification of nucleic acids and/or polypeptides, one may refer to the manufacturer's instructions, when commercial kits or materials, such as for example Qiagen's mini, midi and maxi prep kit or the Macherey-Nagel kit, are used, and/or alternatively refer to the protocols described by J. Sambrook and D. Russell, Molecular Cloning: A Laboratory Manual, 3^{rd} ed., Cold Spring Harbor Laboratory Press, Cold Spring Harbor, N. Y. (2001).

In one embodiment, the produced extra-genomic nucleic acid molecule is released from the genetically modified gram-negative bacterium of the invention, in particular E. coli, preferably by a step of lysis.

The present invention also relates to the use of the genetically modified gram-negative bacterium according to the invention, in particular E. coli, for the production and release from the producing bacteria, of at least one extra-genomic nucleic acid molecule.

The present invention also relates to the use of the genetically modified gram-negative bacterium according to the invention, in particular E. coli, for the production of at least one polypeptide encoded by at least one extra-genomic nucleic acid molecule.

In one embodiment, the produced polypeptide is released from the cytoplasm of the genetically modified gram-negative bacterium according to the invention, in particular E. coli, preferably by a step of cell lysis.

The present invention also relates to the use of the genetically modified gram-negative bacterium according to the invention, in particular E. coli, for the production and release from the producing bacteria, of at least one polypeptide encoded by at least one extra-genomic nucleic acid molecule.

In some embodiments, the genetically modified gram-negative bacterium according to the invention, in particular E. coli, does not produce outer membrane vesicles (OMV). In certain embodiments, the at least one extra-genomic nucleic acid molecule and/or the at least one polypeptide encoded by at least one extra-genomic nucleic acid molecule is/are not released by the means of outer membrane vesicles (OMV).

The present invention also relates to a kit comprising a genetically modified gram-negative bacterium according to the intention, in particular E. coli, and means to transform said bacterium with an extra-genomic nucleic acid molecule.

The invention further relates to a kit comprising a genetically modified E. coli bacterium comprising at least one mutated gene encoding a protein involved in the envelope integrity, said bacterium having an altered envelop integrity and being oversensitive to bacterial lysis as compared to a bacterium with unaltered envelop integrity, wherein the at least one mutated gene is ompA, and/or a homologue thereof, or a gene involved in Lpp functionality; and means to transform said bacterium with an extra-genomic nucleic acid molecule.

In some embodiments, the bacterium from the above kit comprises at least two mutated genes encoding proteins involved in the envelope integrity. In certain embodiments, the bacterium from the above kit comprises at least two mutated genes encoding proteins involved in the envelope integrity, wherein at least one mutated gene is ompA, and/or a homologue thereof, and at least one mutated gene is a gene involved in Lpp functionality.

In some embodiments, the bacterium does not comprise simultaneously a complete deletion of the ompA gene and a complete deletion of the lpp gene.

In one embodiment, bacterium according to the invention is a competent bacterium.

In one embodiment, kit of the invention comprises a plasmid for use as a positive control in a reaction of transformation of the genetically modified gram-negative bacterium of the invention, in particular E. coli.

### Sequences Used Herein

TABLE 1
Sequences used herein

| SEQ ID NO: | Description |
|---|---|
| 1 | ompA nucleic acid sequence (1041 bp) |
| 2 | OmpA preprotein amino acid sequence (346 aa) |
| 3 | OmpA mature protein amino acid sequence (325 aa) |
| 4 | lpp nucleic acid sequence (237 bp) |
| 5 | Lpp preprotein amino acid sequence (78 aa) |
| 6 | Lpp mature protein amino acid sequence (58 aa) |
| 7 | pal nucleic acid sequence (522 bp) |
| 8 | Pal preprotein amino acid sequence (173 aa) |
| 9 | Pal mature protein amino acid sequence (152 aa) |
| 10 | ybiS nucleic acid sequence (921 bp) |
| 11 | YbiS protein amino acid sequence (306 aa) |
| 12 | ycfS nucleic acid sequence (963 bp) |
| 13 | YcfS protein amino acid sequence (320 aa) |
| 14 | erfK nucleic acid sequence (933 bp) |
| 15 | ErfK protein amino acid sequence (310 aa) |
| 16 | yfiB nucleic acid sequence (483 bp) |
| 17 | YfiB protein amino acid sequence (160 aa) |
| 18 | yiaD nucleic acid sequence (660 bp) |
| 19 | YiaD protein amino acid sequence (219 aa) |
| 20 | yqhH nucleic acid sequence (258 bp) |
| 21 | YqhH protein amino acid sequence (85 aa) |

(ompA nucleic acid sequence, 1041 bp)
SEQ ID NO: 1
atgaaaaagacagctatcgcgattgcagtggcactggctggtttcgctaccgtagcgcaggccgctccgaaagataacacct
ggtacactggtgctaaactgggctggtcccagtaccatgacactggtttcatcaacaacaatggcccgacccatgaaaaccaa
ctgggcgctggtgcttttggtggttaccaggttaacccgtatgttggctttgaaatgggttacgactggttaggtcgtatgccgtac
aaaggcagcgttgaaaacggtgcatacaaagctcagggcgttcaactgaccgctaaactgggttacccaatcactgacgacc
tggacatctacactcgtctgggtggcatggtatggcgtgcagacactaaatccaacgtttatggtaaaaaccacgacaccggc
gtttctccggtcttcgctggcggtgttgagtacgcgatcactcctgaaatcgctacccgtctggaataccagtggaccaacaaca
tcggtgacgcacacaccatcggcactcgtccggacaacggcatgctgagcctgggtgtttcctaccgtttcggtcagggcgaa
gcagctccagtagttgctccggctccagctccggcaccggaagtacagaccaagcacttcactctgaagtctgacgttctgttc
aacttcaacaaagcaaccctgaaaccggaaggtcaggctgctctggatcagctgtacagccagctgagcaacctggatccga
aagacggttccgtagttgttctgggttacaccgaccgcatcggttctgacgcttacaaccagggtctgtccgagcgccgtgctc
agtctgttgttgattacctgatctccaaaggtatcccggcagacaagatctccgcacgtggtatgggcgaatccaacccggttac
tggcaacacctgtgacaacgtgaaacagcgtgctgcactgatcgactgcctggctccggatcgtcgcgtagagatcgaagtta
aaggtatcaaagacgttgtaactcagccgcaggcttaa
(OmpA preprotein amino acid sequence, 346 aa)
SEQ ID NO: 2
MKKTAIAIAVALAGFATVAQAAPKDNTWYTGAKLGWSQYHDTGFINNNGPTH
ENQLGAGAFGGYQVNPYVGFEMGYDWLGRMPYKGSVENGAYKAQGVQLTA
KLGYPITDDLDIYTRLGGMVWRADTKSNVYGKNHDTGVSPVFAGGVEYAITPE
IATRLEYQWTNNIGDAHTIGTRPDNGMLSLGVSYRFGQGEAAPVVAPAPAPAP
EVQTKHFTLKSDVLFNFNKATLKPEGQAALDQLYSQLSNLDPKDGSVVVLGYT
DRIGSDAYNQGLSERRAQSVVDYLISKGIPADKISARGMGESNPVTGNTCDNVK
QRAALIDCLAPDRRVEIEVKGIKDVVTQPQA
(OmpA mature protein amino acid sequence, 325 aa)
SEQ ID NO: 3
APKDNTWYTGAKLGWSQYHDTGFINNNGPTHENQLGAGAFGGYQVNPYVGF
EMGYDWLGRMPYKGSVENGAYKAQGVQLTAKLGYPITDDLDIYTRLGGMVW
RADTKSNVYGKNHDTGVSPVFAGGVEYAITPEIATRLEYQWTNNIGDAHTIGT
RPDNGMLSLGVSYRFGQGEAAPVVAPAPAPAPEVQTKHFTLKSDVLFNFNKAT
LKPEGQAALDQLYSQLSNLDPKDGSVVVLGYTDRIGSDAYNQGLSERRAQSVV
DYLISKGIPADKISARGMGESNPVTGNTCDNVKQRAALIDCLAPDRRVEIEVKGI
KDVVTQPQA
(lpp nucleic acid sequence, 237 bp)
SEQ ID NO: 4
atgaaagctactaaactggtactgggcgcggtaatcctgggttctactctgctggcaggttgctccagcaacgctaaaatcgat
cagctgtcttctgacgttcagactctgaacgctaaagttgaccagctgagcaacgacgtgaacgcaatgcgttccgacgttcag
gctgctaaagatgacgcagctcgtgctaaccagcgtctggacaacatggctactaaataccgcaagtaa
(Lpp preprotein amino acid sequence, 78 aa)
SEQ ID NO: 5
MKATKLVLGAVILGSTLLAGCSSNAKIDQLSSDVQTLNAKVDQLSNDVNAMRS
DVQAAKDDAARANQRLDNMATKYRK
(Lpp mature protein amino acid sequence, 58 aa)
SEQ ID NO: 6
CSSNAKIDQLSSDVQTLNAKVDQLSNDVNAMRSDVQAAKDDAARANQRLDN
MATKYRK
(pal nucleic acid sequence, 522 bp)
SEQ ID NO: 7
atgcaactgaacaaagtgctgaaagggctgatgattgctctgcctgttatggcaattgcggcatgttcttccaacaagaacgcca
gcaatgacggcagcgaaggcatgctgggtgccggcactggtatggatgcgaacggcggcaacggcaacatgtcttccgaa
gagcaggctcgtctgcaaatgcaacagctgcagcagaacaacatcgtttacttcgatctggacaagtacgatatccgttctgac
ttcgctcaaatgctggatgcacatgcaaacttcctgcgtagcaacccgtcttacaaagtcaccgtagaaggtcacgcggacga
acgtggtactccggaatacaacatctccctgggtgaacgtcgtgcgaacgccgttaagatgtacctgcagggtaaaggcgttt
ctgcagaccagatctccatcgtttcttacggtaaagaaaaacctgcagtactgggtcatgacgaagcggcatactccaaaaacc
gtcgtgcggtactggtttactaa
(Pal preprotein amino acid sequence, 173 aa)
SEQ ID NO: 8
MQLNKVLKGLMIALPVMAIAACSSNKNASNDGSEGMLGAGTGMDANGGNGN
MSSEEQARLQMQQLQQNNIVYFDLDKYDIRSDFAQMLDAHANFLRSNPSYKV
TVEGHADERGTPEYNISLGERRANAVKMYLQGKGVSADQISIVSYGKEKPAVL
GHDEAAYSKNRRAVLVY
(Pal mature protein amino acid sequence, 152 aa)
SEQ ID NO: 9
CSSNKNASNDGSEGMLGAGTGMDANGGNGNMSSEEQARLQMQQLQQNNIVY
FDLDKYDIRSDFAQMLDAHANFLRSNPSYKVTVEGHADERGTPEYNISLGERR
ANAVKMYLQGKGVSADQISIVSYGKEKPAVLGHDEAAYSKNRRAVLVY
(ybiS nucleic acid sequence, 921 bp)
SEQ ID NO: 10
atgaatatgaaattgaaaacattattcgcagcggccttcgctgttgtcggcttttgcagtaccgcctctgcggtaacttatcctctg
ccaaccgacgggagtcgcctggttggtcagaatcaggtgatcaccattcctgaaggtaacactcagccgctggagtattttgcc
gccgagtaccagatggggctttccaatatgatggaagcgaacccgggtgtggataccttcctgccgaaaggcggtactgtact
gaacattccgcagcagctgatcctgccggataccgttcatgaaggcatcgtcattaacagtgctgagatgcgtctttattactatc
cgaaagggaccaacaccgttatcgtgctgccgatcggcattggtcagttaggcaaagatacgcctatcaactggaccaccaa
agttgagcgtaaaaaagcaggcccgacctggacgccgaccgccaaaatgcacgcagagtaccgcgctgcgggcgaaccg
cttccggctgtcgttccggcaggtccggataacccgatggggctgtatgcactctatatcggtcgcctgtatgctatccatggca
ccaacgccaacttcggtatcggcctgcgtgtaagtcatggttgtgtgcgtctgcgtaacgaagacatcaaattcctgttcgagaa
agtaccggtcggtacccgcgtacagtttattgatgagccggtaaaagcgaccaccgagccagacggcagccgttatattgaa
gtccataacccgctgtctaccaccgaagcccagtttgaaggtcaggaaattgtgccaattaccctgacgaagagcgtgcagac
agtgaccggtcagccagatgttgaccaggttgttcttgatgaagcgattaaaaaccgctccgggatgccggttcgtctgaattaa
(YbiS protein amino acid sequence, 306 aa)
SEQ ID NO: 11
MNMKLKTLFAAAFAVVGFCSTASAVTYPLPTDGSRLVGQNQVITIPEGNTQPLE
YFAAEYQMGLSNMMEANPGVDTFLPKGGTVLNIPQQLILPDTVHEGIVINSAE
MRLYYYPKGTNTVIVLPIGIGQLGKDTPINWTTKVERKKAGPTWTPTAKMHAE
YRAAGEPLPAVVPAGPDNPMGLYALYIGRLYAIHGTNANFGIGLRVSHGCVRL
RNEDIKFLFEKVPVGTRVQFIDEPVKATTEPDGSRYIEVHNPLSTTEAQFEGQEIV
PITLTKSVQTVTGQPDVDQVVLDEAIKNRSGMPVRLN
(ycfS nucleic acid sequence, 963 bp)
SEQ ID NO: 12
gtgatgatcaaaacgcgtttttctcgctggctaacgttttttacgttcgccgctgccgtggcgctggcgctaccggcaaaagcca
acacctggccgctgccgccagcgggcagtcgtctggttggcgaaaacaaatttcatgtggtggaaaatgacggtggttctctg
gaagccatcgccaaaaaatacaacgtcggctttctcgctctgttacaggctaaccccggcgttgatccttacgtaccgcgcgcg
ggcagcgtgttaacgatcccgttgcaaaccctacttccagatgcgccgcgcgaaggcattgtgatcaacattgcggagctgcg
tctctattactacccgccgggtaaaaattcggtaaccgtgtatccaataggtattggtcagttaggtggtgacacgctgacaccg
acaatggtgaccaccgtttcagacaaacgtgcaaacccaacctggacgccaacggcaaacatccgcgcccgttataaagca
cagggaattgagttgcctgcggtagtgccggctggactggataacccaatgggccatcatgcgattcgtctggcggcctatgg
cggcgtttatttgcttcatggtacgaacgccgatttcggcattggcatgcgggtaagttctggctgtattcgtctgcgggatgacg
atatcaaaacactctttagccaggtcaccccaggcaccaaagtgaatatcatcaacactccgataaaagtctctgccgaaccaa
acggtgcgcgtctggttgaagtacatcagccgctgtcagagaagattgatgacgatccgcagctgctgccaattacgctgaat
agcgcaatgcaatcatttaaagatgcagcacaaactgacgctgaagtgatgcaacatgtgatggatgtccgttccgggatgcc
ggtggatgtccgccgtcatcaagtgagcccacaaacgctgtaa
(YefS protein amino acid sequence, 320 aa)
SEQ ID NO: 13
VMIKTRFSRWLTFFTFAAAVALALPAKANTWPLPPAGSRLVGENKFHVVENDG
GSLEAIAKKYNVGFLALLQANPGVDPYVPRAGSVLTIPLQTLLPDAPREGIVINI
AELRLYYYPPGKNSVTVYPIGIGQLGGDTLTPTMVTTVSDKRANPTWTPTANIR
ARYKAQGIELPAVVPAGLDNPMGHHAIRLAAYGGVYLLHGTNADFGIGMRVS
SGCIRLRDDDIKTLFSQVTPGTKVNIINTPIKVSAEPNGARLVEVHQPLSEKIDDD
PQLLPITLNSAMQSFKDAAQTDAEVMQHVMDVRSGMPVDVRRHQVSPQTL
(erfK nucleic acid sequence, 933 bp)
SEQ ID NO: 14
atgcgtcgtgtaaatattctttgctcatttgctctgctttttgccagccatactagcctggcggtaacttatccattacctccagaggg
tagccgtttagtggggcagtcgtttactgtaactgttcctgatcacaatacccagccgctggagacttttgccgcacaatacggg
caagggttaagtaacatgctggaagcgaacccgggcgctgatgtttttttgccgaagtctggctcgcaactcaccattccgcag
caactgattttgcccgacactgttcgtaaagggattgttgttaacgtcgctgagatgcgtctttattactacccaccagacagtaat
actgtggaagtctttcctattggtatcggccaggctgggcgagaaaccccgcgtaactgggtgactaccgttgaacgtaaaca
agaagctccaacctggacgccaacgccgaacactcggcgcgaatatgcgaaacgaggggagagtttgcccgcatttgttcct
gcgggccccgataatcccatggggctgtacgcgatttatattggcaggttgtatgccatccatggtaccaatgccaattttggtat
tgggctccgggtaagtcagggctgtattcgtctgcgcaatgacgatatcaaatatctgtttgataatgttcctgttgggacgcgtgt
gcaaattatcgaccagccagtaaaatacaccactgaaccagatggctcgaactggctggaagttcatgagccattgtcgcgca
atcgtgcagaatatgagtctgaccgaaaagtgccattgccggtaaccccatctttgcgggcgtttatcaacgggcaagaagttg
atgtaaatcgcgcaaatgctgcgttgcaacgtcgatcgggaatgcctgtgcaaattagttctggttcaagacagatgttttaa
(ErfK protein amino acid sequence, 310 aa)
SEQ ID NO: 15
MRRVNILCSFALLFASHTSLAVTYPLPPEGSRLVGQSFTVTVPDHNTQPLETFAA
QYGQGLSNMLEANPGADVFLPKSGSQLTIPQQLILPDTVRKGIVVNVAEMRLY
YYPPDSNTVEVFPIGIGQAGRETPRNWVTTVERKQEAPTWTPTPNTRREYAKRG
ESLPAFVPAGPDNPMGLYAIYIGRLYAIHGTNANFGIGLRVSQGCIRLRNDDIKY
LFDNVPVGTRVQIIDQPVKYTTEPDGSNWLEVHEPLSRNRAEYESDRKVPLPVT
PSLRAFINGQEVDVNRANAALQRRSGMPVQISSGSRQMF
(yfiB nucleic acid, 483 bp)
SEQ ID NO: 16
atgataaagcacctggtagcacccctggttttcacctcactaatactgactggctgccagtcccctcagggaaagtttactcctg
agcaagtcgccgctatgcaatcttatggatttactgaatccgccggcgactggtcgctgggcttatcagatgccattctgttcgca
aaaaatgactacaaattgctcccggaaagccagcaacagatccaaaccatggcagctaaattggcctcgacagggctaacac
atgcccgtatggatggacacaccgataactatggtgaagacagttacaacgaaggcttatcattgaaacgggcgaatgtcgtg
gccgatgcatgggctatgggtggacaaattccacgcagcaatctcaccacacagggtttaggaaaaaaatatcccatagcca
gtaacaagaccgcccagggccgcgccgagaaccgccgcgtcgcagtggtgattactaccccttaa
(YfiB protein amino acid sequence, 160 aa)
SEQ ID NO: 17
MIKHLVAPLVFTSLILTGCQSPQGKFTPEQVAAMQSYGFTESAGDWSLGLSDAI
LFAKNDYKLLPESQQQIQTMAAKLASTGLTHARMDGHTDNYGEDSYNEGLSL
KRANVVADAWAMGGQIPRSNLTTQGLGKKYPIASNKTAQGRAENRRVAVVIT
TP
(yiaD nucleic acid, 660 bp)
SEQ ID NO: 18
atgaagaaacgtgtttatcttattgccgccgtagtgagtggcgctctggcggtatctggctgcacaactaacccttacaccggcg
aacgcgaagcaggtaaatctgctatcggcgcaggtctgggctctctcgtgggcgcgggtattggtgcgctctcttcttcgaaga
aagatcgcggtaaaggcgcgctgattggcgcagcagcaggcgcagctctgggcggcggcgttggttattacatggatgtgc
aggaagcgaagctgcgcgacaaaatgcgcggcactggtgttagcgtaacccgcagcggggataacattatcctcaatatgcc
gaacaatgtgaccttcgacagcagcagcgcgaccctgaaaccggcgggcgctaacaccctgaccggcgtggcaatggtac
tgaaagagtatccgaaaacggcggttaacgtgattggttataccgacagcacgggtggtcacgacctgaacatgcgtctctcc
cagcaacgtgcggattccgttgccagcgcgttgatcacccagggcgtggacgccagccgcatccgtactcagggccttggc
ccggctaacccaatcgccagcaacagcaccgcagaaggtaaggcgcaaaaccgccgtgtagaaattaccttaagcccgctg
taa
(YiaD protein amino acid sequence, 219 aa)
SEQ ID NO: 19
MKKRVYLIAAVVSGALAVSGCTTNPYTGEREAGKSAIGAGLGSLVGAGIGALS
SSKKDRGKGALIGAAAGAALGGGVGYYMDVQEAKLRDKMRGTGVSVTRSGD
NIILNMPNNVTFDSSSATLKPAGANTLTGVAMVLKEYPKTAVNVIGYTDSTGG
HDLNMRLSQQRADSVASALITQGVDASRIRTQGLGPANPIASNSTAEGKAQNR
RVEITLSPL
(yqhH nucleic acid, 258 bp)
SEQ ID NO: 20
atgaaaacgattttcaccgtgggagctgttgttctggcaacctgcttgctcagtggctgcgtcaatgagcaaaaggtcaatcagc
tggcgagcaatgtgcaaacattaaatgccaaaatcgcccggcttgagcaggatatgaaagcactacgcccacaaatctatgct
gccaaatccgaagctaacagagccaatacgcgtcttgatgctcaggactattttgattgcctgcgctgcttgcgtatgtacgcag
aatga
(YqhH protein amino acid sequence, 85 aa)
SEQ ID NO: 21
MKTIFTVGAVVLATCLLSGCVNEQKVNQLASNVQTLNAKIARLEQDMKALRP
QIYAAKSEANRANTRLDAQDYFDCLRCLRMYAE

### BRIEF DESCRIPTION OF THE DRAWINGS

FIG. 1 is a photograph showing the bacterial survival upon an osmotic shock with pure water of a wild type MG1655 E. coli strain (WT; control), and MG1655 E. coli strains having the following genotypes ΔompA, Δlpp or Δpal. Bacterial dilutions are indicated at the bottom of the figure.

FIG. 2 is a photograph showing the bacterial survival upon an osmotic shock with water of a wild type MG1655 E. coli strain (WT; control), and MG1655 E. coli strains having the following genotypes ompAR256E or lppΔK58 or both ompAR256E and lppΔK58. Bacterial dilutions are indicated at the bottom of the figure.

FIG. 3 is a photograph showing the bacterial survival upon an osmotic shock with water of a wild type MG1655 E. coli strain (WT; control), and an MG1655 E. coli strain having the following genotype ompAD241N or both ompAD241N and Δlpp. Bacterial dilutions are indicated at the bottom of the figure.

FIG. 4 is a photograph showing the bacterial survival upon an osmotic shock with water of a wild type MG1655 E. coli strain (WT; control), MG1655 E. coli strain having a ompAR256E and lppΔK58 genotype (control), and MG1655 E. coli strains having a combination of mutations among ompAR256E, ΔybiS, ΔycfS, ΔerfK. Bacterial dilutions are indicated at the bottom of the figure.

FIG. 5 is a photograph showing the bacterial survival upon an osmotic shock with water of a wild type MG1655 E. coli strain (WT; control), and MG1655 E. coli strains having a combination of mutations among lppR57L, ompAR256E, ΔybiS, ΔycfS, ΔerfK. Bacterial dilutions are indicated at the bottom of the figure.

FIG. 6 is a photograph showing the bacterial survival upon an osmotic shock with water of a wild type MG1655 E. coli strain (control), and an MG1655 E. coli strain having the following genotype lppΔK58 or ompAR256E or Δpal or lppΔK58 ompAR256E or lppΔK58 Δpal or ompAR256E Δpal. Bacterial dilutions are indicated at the bottom of the figure.

FIG. 7 is a photograph showing the bacterial survival upon an osmotic shock with water (A), without osmotic shock in PBS buffer (B) or LB miller culture medium (C) of wild type MG1655 E. coli strain (control) and MG1655 E. coli strain having the following genotypes Δlpp or Δlpp ΔompA or Δlpp ompAΔCter. Dilutions (10⁻² and 10⁻³) are indicated on the right side of the photograph.

FIG. 8 is a photograph showing the analysis by electrophoresis of plasmidic DNA recovered from a wild type MG1655 E. coli strain (lane 1) and MG1655 E. coli strain of genotype ompAR256E lppΔK58 (lane 2) after a preparation protocol using non-commercial lysis buffers (home-made).

FIG. 9 is a photograph showing the analysis by electrophoresis of plasmidic DNA recovered from a control MG1655 E. coli strain (lanes 1 and 2) and MG1655 E. coli strain of genotype ompAR256E lppΔK58 (lanes 3 and 4) after a preparation protocol using Qiagen® Maxi prep kit using either the recommended volumes for the P1, P2 and P3 buffers (lanes 1 and 3) or dividing said volumes by 4 (lanes 2 and 4).

FIG. 10 is a photograph showing the analysis by electrophoresis of plasmidic DNA recovered from a control DH10B E. coli strain (Lane 1), DH10B E. coli strain of genotype ompAR256E lppΔK58 (lane 2), control DH5α E. coli strain (Lane 3) and DH5α E. coli strain of genotype ompAR256E lppΔK58 (lane 4). The arrow indicates the population of plasmids corresponding to the super-coiled form.

### EXAMPLES

The present invention is further illustrated by the following examples.

### Example 1: Bacterial Strains Used in the Study

### 1—Materials and Methods

### 1.1—Recipient Strains

The list of recipient strains which were used to engineer the mutations are given in Table 2.

TABLE 2
list of recipient strains

| Strain (WT) | Genotype |
|---|---|
| E. coli MG1655 | F⁻ λ⁻ ilvG⁻ rfb-50 rph-1 |
| E. coli DH5α | F⁻, Δ(argF-lac)169, φ80dlacZ58(M15), |
| | ΔphoA8, glnX44(AS), λ⁻, |
| | deoR481, rfbC1?, gyrA96(NalR), recA1, |
| | endA1, thiE1, hsdR17 |
| E. coli DH10B | F⁻ mcrA Δ(mrr-hsdRMS-mcrBC) φ80lacZΔM15 |
| | ΔlacX74 recA1 endA1 araD139 Δ(ara-leu)7697 |
| | galU galK λ⁻ rpsL(Str^{R}) nupG |

### 1.2—Deletions

MG1655 E. coli strain was used as the recipient strain. P1 lysate of ompA772(del)::kan or lpp-752(del)::kan or pal-790(del)::kan or ybiS790(del)::kan or ycfS775(del)::kan or erfK761(del)::kan obtained from the Keio strain collection (Baba et al., Mol Syst Biol. 2006; 2:2006.0008) were used as donors and transduced in the recipient strain as described in Thomason et al. (Curr Protoc Mol Biol. 2007 July; Chapter 1:Unit 1.17). The simple mutants were selected on LB/agar plate containing kanamycin. The gene encoding for kanamycin resistance was then excised by FLP recombinase. Then, other P1 transduction were performed into the first backgrounds to create the combined deletion strains.

### 1.3—Point Mutations and Partial Deletions

Recombination with ds-DNA were performed in E. coli MG1655 using the Lambda-Red system as reported in Thomason et al. (Plasmid. 2007 September; 58(2):148-58).

We used a two-step recombination method, first using cat-sacB integrated at the desired site and selected on chloramphenicol, followed by a second Lambda-Red recombination to replace the cat-sacB by the final mutated locus of choice without modifying the surrounding DNA region.

For the ompA mutants, the PCR product ompA::cat-sacB was integrated via a first Lambda-Red recombination followed by a second Lambda-Red recombination with ompA::ompAR256E or ompA::ompAD241N or ompA::ompAΔc. For lpp mutants, lpp::cat-sacB was first integrated and then counter-selected after recombination with lpp::lppΔK58 or lpp::lppK58R.

### 1.4—Strains

For plasmid extraction assays, we integrated a Kanamycin resistant gene (KanR) by Lambda-Red recombination between the genes fumD and pikF of the strain lpp::lppΔK58. We also integrated a Kanamycin resistant gene (KanR) by Lambda-Red recombination between the genes ompA and matP of the strain ompA::ompAR256E. The sequences lpp::/lppΔK58 fumD-kanR-PikF (lppΔK58-KanR) and ompA:: ompAR256E-d kanR-matP (ompaR256E-ΔKanR) were introduced using P1 transduction in strains E. coli DH5α and E. coli DH10B.

The strains used in this study and their genotype are indicated in Table 3.

TABLE 3
E. coli strains used in this study

| | | Recipient |
|---|---|---|
| Mutant | Mutations | strain used |
| ΔompA | ompA772 (del) | E. coli MG1655 |
| ompAR256E | ompA::ompAR256E | E. coli MG1655 |
| lppΔK58 | lpp::lppΔK58 | E. coli MG1655 |
| lppΔK58 ompAR256E | lpp::lppΔK58 ompA::ompAR256E | E. coli MG1655 |
| Δlpp ompAD241N | lpp-752(del) ompA::ompAD241N | E. coli MG1655 |
| ompAD241N | ompA::ompAD241N | E. coli MG1655 |
| ompAR256E ΔybiS | ompA::ompAR256E ybiS790(del) | E. coli MG1655 |
| ompAR256E ΔycfS | ompA::ompAR256E ycfS775(del) | E. coli MG1655 |
| ompAR256E ΔerfK | ompA::ompAR256E erfK761(del) | E. coli MG1655 |
| ompAR256E ΔybiS | ompA::ompAR256E ybiS790(del) | E. coli MG1655 |
| ΔycfS | ycfS775(del) | |
| ompAR256E ΔybiS | ompA::ompAR256E ybiS790(del) | E. coli MG1655 |
| ΔerfK | erfK761(del) | |
| ompAR256E ΔybiS | ompA::ompAR256E ybiS790(del) | E. coli MG1655 |
| ΔycfS ΔerfK | ycfS775(del) erfK761(del) | |
| lppR57L | lpp::lppR57L | E. coli MG1655 |
| lppR57L ompAR256E | lpp::lppR57L ompA::ompAR256E | E. coli MG1655 |
| lppR57L ompAR256E | lpp::lppR57L ompA::ompAR256E | E. coli MG1655 |
| ΔybiS | ybiS790(del) | |
| lppR57L ompAR256E | lpp::lppR57L ompA::ompAR256E | E. coli MG1655 |
| ΔycfS | ycfS775(del) | |
| lppR57L ompAR256E | lpp::lppR57L ompA::ompAR256E | E. coli MG1655 |
| ΔerfK | erfK761(del) | |
| lppR57L ompAR256E | lpp::lppR57L ompA::ompAR256E | E. coli MG1655 |
| ΔybiS ΔerfK | ybiS790(del) erfK761(del) | |
| lppR57L ompAR256E | lpp::lppR57L ompA::ompAR256E | E. coli MG1655 |
| ΔybiS ΔycfS | ybiS790(del) ycfS775(del) | |
| lppR57L ompAR256E | lpp::lppR57L ompA::ompAR256E | E. coli MG1655 |
| ΔycfS ΔerfK | ycfS775(del) erfK761(del) | |
| lppR57L ompAR256E | lpp::lppR57L ompA::ompAR256E | E. coli MG1655 |
| ΔybiS ΔycfS ΔerfK | ybiS790(del) ycfS775(del) erfK761(del) | |
| Δpal | pal-790(del) | E. coli MG1655 |
| lppAK58 Δpal | lpp::lppΔK58 pal-790(del) | E. coli MG1655 |
| ompAR256E Δpal | ompA::ompAR256E pal-790(del) | E. coli MG1655 |
| Δlpp | lpp-752(del) | E. coli MG1655 |
| Δlpp ΔompA | lpp-752(del) ompA772(del) | E. coli MG1655 |
| Δlpp ompAΔCter | lpp-752(del) ompA::ompAΔCter | E. coli MG1655 |
| lppΔK58 ompAΔCter | lpp::lppΔK58-KanR | E. coli MG1655 |
| | ompA::ompAΔCter | |
| lppΔK58 ompAR256E | lpp::lppΔK58-KanR | E. coli DH5α |
| | ompA::ompAR256E-KanR | |
| lppΔK58 ompAR256E | lpp::lppΔK58-KanR | E. coli DH10B |
| | ompA::ompAR256E-KanR | |
| ompAR256E | ompA::ompAR256E | E. coli DH10B |
| lppΔK58 | lpp::lppΔK58 | E. coli DH10B |
| lppΔK58 ompAR256E | lpp::lppΔK58 ompA::ompAR256E | E. coli DH10B |

### 2—Results

### 2.1—ompA Mutations

The OmpA protein spans across the outer membrane of the bacterial envelope of gram-negative bacteria thanks to its N-terminal β-barrel. The soluble C-terminal portion of the protein extends inside the periplasm and interact non-covalently with the periplasmic peptidoglycan. To interfere with the interaction between the outer membrane and the periplasmic peptidoglycan, the following ompA mutations were used, (i) a complete deletion of the ompA gene (ΔompA or ompA772(del))—(Baba et al., Mol Syst Biol. 2006; 2:2006.0008), and (ii) point mutations in the residues mediating the interaction of ompA with periplamic peptidoglycan (Ishida et al., Biochim Biophys Acta. 2014 December; 1838(12):3014-24): the codon encoding Arginine (R) at position 256 in SEQ ID NO: 3 was substituted with a codon encoding a Glutamic acid (E)—ompA::ompAR256E; the codon encoding Aspartic acid (D) at position 241 in SEQ ID NO: 3 was substituted with a codon encoding an Asparagine (N)—ompA::ompAD241N. A partial deletion of the C-terminal portion, consisting of amino acid 171 to amino acid 325 in SEQ ID NO: 3 was also generated—ompA::ompAΔCter. The ompA::ompAR256E creates a negative charge at residue 256 while it was previously positively charged. This creates an electrostatic repulsion between the mutated OmpA protein and the peptidoglycan that abolishes their interaction. The ompA::ompAD241N abolishes the charge at residue 241 and therefore the interaction with the peptidoglycan.

### 2.2—Lpp Mutations

The Lpp protein in E. coli tethers the outer membrane of the bacterial envelope to the periplasmic peptidoglycan. The protein is anchored via its lipidated N-terminus to the outer membrane and attached via its C-terminal lysine to the short peptidic backbone present in periplasmic peptidoglycan. To interfere with the interaction between the outer membrane and the periplasmic peptidoglycan, the following lpp mutation were used, (i) a complete deletion of the lpp gene—lpp-752(del)—(Baba et al., Mol Syst Biol. 2006; 2:2006.0008), and (ii) point mutations affecting the interaction of Lpp with periplasmic peptidoglycans (Zhang et al., J Biol Chem. 1992 Sep. 25; 267(27):19560-4). The deletion of the codon encoding lysine (K) at position 58 Lpp protein of sequence SEQ ID NO: 6 mediating the interaction of Lpp with periplasmic peptidoglycans was deleted—lpp::lppΔK58; the codon encoding Arginine (R) at position 57 of SEQ ID NO: 6 was substituted with a codon encoding a Leucine (L)—lpp::lppR57L. In the lpp::lppR57L mutant, the lysine essential for the cross-linking of Lpp to peptidoglycan is still present but the adjacent residue has been modified. This modification lowers the binding of Lpp to peptidoglycan by 70% when compared to a wild type Lpp protein.

### 2.3—ybiS, ycfS and erfK Mutations

These 3 genes each encode enzymes catalyzing the covalent binding of the mature Lpp protein via its C-terminal lysine to the periplasmic peptidoglycan. To interfere with the interaction between the outer membrane and the periplasmic peptidoglycan the following mutations were used: a complete deletion of the ybiS gene—ybiS790(del)-, a complete deletion of the ycfS gene—ycf5775(del)—and a completed deletion of the erfK gene—erfK761(del)—(Baba et al., Mol Syst Biol. 2006; 2:2006.0008).

### 2.4—pal Mutation

The Pal protein is a lipoprotein that belongs to the Tol-Pal constriction apparatus. It participates in the attachment between the outer membrane and periplasmic peptidoglycan in the bacterial envelope. To interfere with the interaction between the outer membrane and the periplasmic peptidoglycan the following mutations was used: a complete deletion of the pal gene—pal-790(del)—(Baba et al., Mol Syst Biol. 2006; 2:2006.0008).

### Example 2. Effect of Mutation(s) Affecting the Bacterial Cell Wall on Survival to Osmotic Shocks

### 1—Materials and Methods

### 1—Bacterial Culture

E. coli strains were grown in LB culture medium (1% tryptone, 0.5% yeast extract, 1% NaCl, pH 7.0-7.2) at 37° C. under agitation. Whenever necessary, antibiotics were used at 25 μg/ml (e.g. Kanamycin).

1.2—Quantification of Survival after Osmotic Shock

Exponential or stationary culture phase E. coli strains were pelleted and resuspended in twice their initial volume with pure water for 5 minutes. This resuspension was then serially diluted (10^{th} dilutions) in water and directly spotted on LB/agar plate incubated overnight at 37° C.

### 2—Results

First, survival of E. coli strain MG1655 with a deletion mutation of ompA, lpp or pal was assessed upon osmotic shock and compared to a wild type MG1655 strain. FIG. 1 shows that the survival of deletion mutants after an osmotic shock is almost comparable to the wild type strain. In other words, single mutation in ompA, lpp or pal genes only slightly affect the sensitivity of E. coli strains to an osmotic shock.

After osmotic shock, survival of E. coli strain MG1655 with the mutation ompAR256E was similar to that observed in control. Survival after osmotic shock of E. coli strain MG1655 with the mutation lppΔK58 decreased by a factor of about 10 when compared to control.

Therefore, single mutations ompAR256E and lppΔK58 only slightly affect the envelop integrity with respect to sensitivity to osmotic shock.

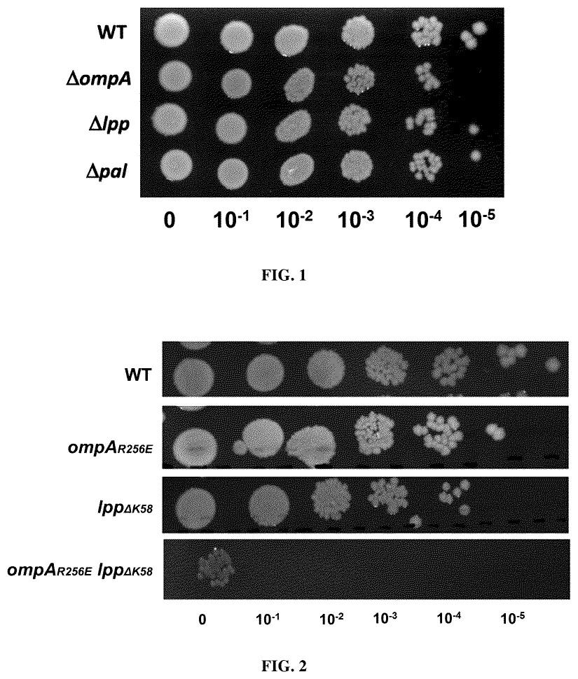

In contrast, survival after osmotic shock of E. coli strain MG1655 with the mutations ompAR256E lppΔK58 sharply decreased by a factor 10⁵ when compared to control and decreased by a factor below 10⁴ when compared to the lppΔK58 single mutant (FIG. 2).

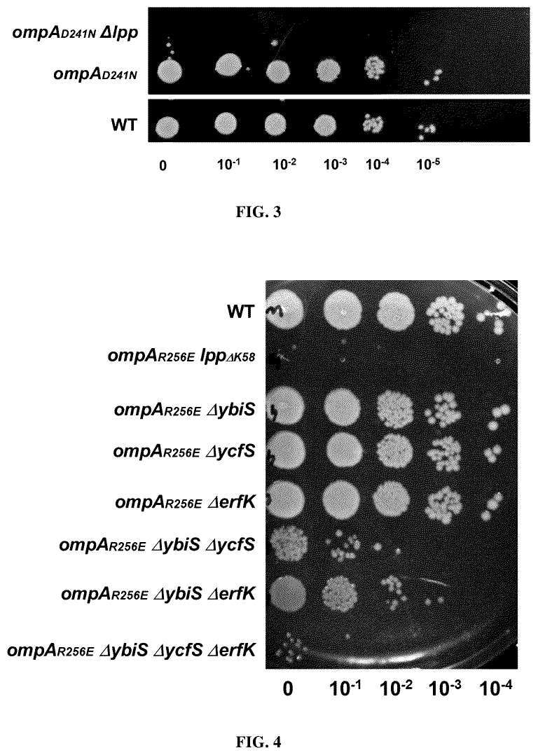

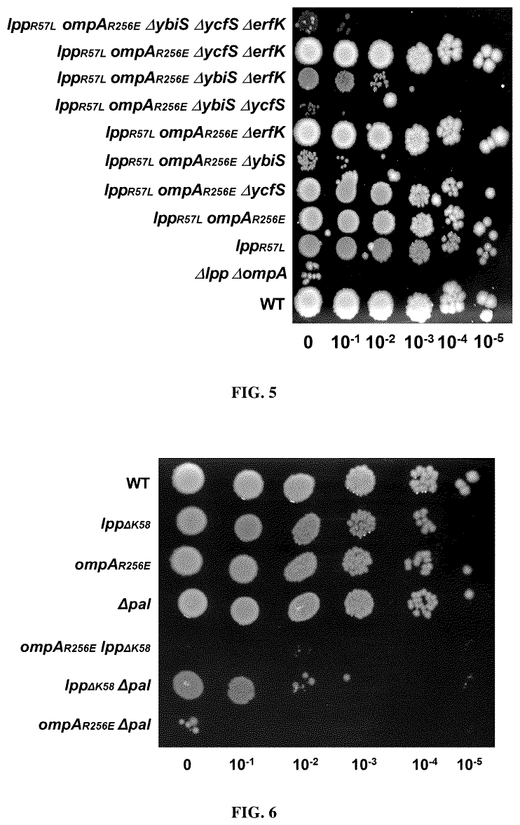

Similarly, whereas the single ompAD241N mutation had no effect towards the bacterial survival upon osmotic shock when compared to the control bacteria, combination of the ompAD241N mutation with a deletion of the lpp gene (Δlpp) affected the survival after osmotic shock by a factor 10⁶ (FIG. 3). The survival after osmotic shock of E. coli strain MG1655 with the mutations ompAR256E ΔybiS ΔycfS ΔerfK decreased by a factor below 10⁴ when compared to control (FIG. 4). The survival after osmotic shock of E. coli strain MG1655 having the genotype lppR77L ompAR256E ΔybiS ΔerfK decreased by a factor of about 10³ when compared to control. The survival after osmotic shock of E. coli strain MG1655 having the genotype lppR57L ompAR256E ΔybiS ΔycfS decreased by a factor of about 10⁵ when compared to control (FIG. 5).

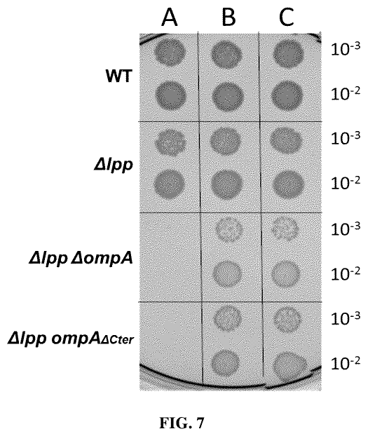

The combination of the mutation Δpal with ompAR256E leads to a decrease in the survival after osmotic shock of E. coli strain MG1655 by a factor of about 10⁵ when compared to control. The combination of the mutation Δpal with lppΔK58 leads to a decrease in the survival after osmotic shock by a factor of about 10² when compared to control. This observation is in contrast with the survival after osmotic shock of the single Δpal mutant that was similar to control (FIG. 6). The survival after osmotic shock of E. coli strain MG1655 with the mutation Δlpp mutant was similar to control. The survival after osmotic shock of E. coli strain MG1655 with the mutations Δlpp ΔompA or Δlpp ompAΔc was lower than the survival of the single Δlpp mutant. Of note, the conditions without osmotic shock showed that the density of the colonies of E. coli strain MG1655 having the genotype Δlpp ΔompA or Δlpp ompAΔc was feinter when compared to control or to the single Δlpp mutant (FIG. 7).

### 3—Conclusion

Significant decrease in survival after osmotic shock was observed in MG1655 E. coli strain bearing the combination of mutations lppΔK58 ompAR256E; Δlpp ompAD241N; ompAR256E ΔybiS ΔycfS ΔerfK: lppR57L ompAR256E ΔybiS ΔycfS; ompAR256E Δpal. These strains are therefore oversensitive to an osmotic shock when compared to reference bacteria.

In addition, MG1655 E. coli strain bearing the combination of mutations lppR57L ompAR256E ΔybiS ΔerfK or lppΔK58 Δpal or Δlpp ΔompA or Δlpp ompAΔc also displayed a decrease of survival following osmotic shock when compared to single mutants or control, although this decrease was more modest when compared to the mutants above.

Combining at least two mutations affecting the interaction between the outer membrane and the periplasmic peptidoglycans leads to the synergistic decrease of E. coli survival after osmotic shock.

### Example 3. The use of E. coli strain MG1655 of genotype lppΔK58 ompAR256E

increases the recovery of extrachromosomal DNA

### 1—Materials and Methods

### 1.1—Bacterial Culture and Preparation of Competent Bacteria

Exponential phase culture grown in LB (1% tryptone, 0.5% yeast extract, 1% NaCl) at 37° C. under agitation were washed several times in cold Water supplemented with Sorbitol and kept on ice before electroporation. Whenever necessary, antibiotics were used at 25 μg/mL (kanamycin; Kan), 15 μg/mL (chloramphenicol; Cm) or 100 μg/mL (ampicillin; Amp).

Recovery of Plasmidic DNA:

The low copy plasmid pBAD18-Cm (pBAD18-Cm (ATCC® 87396™) was transformed into either a control of MG1655 E. coli strain or an MG1655 E. coli strain with the mutations lppΔK58 ompAR256E using the protocol of preparation of plasmid DNA by alkaline lysis with SDS (Molecular cloning: A laboratory manual. Green and Sambrook).

For small scale preparation, 1 mL of bacterial culture was further treated as follows:
Centrifuge 1 ml of culture.
Proceed to lysis
   add 150 μL of P1 (50 mM Tris-HCL, 80 mM EDTA, 100 μg/Ml RNase A, pH 8.0; kept on ice);
   Add 150 μL of P2 (100 mM NaOH, 1% SDS);
   Mix well by inverting the tube several times;
   Incubate for 5 min at room temperature;
   Add 300 μL of P3 (3 M KAc, pH 8.0; kept on ice).
Keep 10 min on ice;
Centrifuge 12,500 rpm 15 min at 4° C.;
Keep supernatant;
Add 0.7 volume of isopropanol (˜630 μL) and gently flip the tube several time;
Centrifuge 12,500 rpm 15 min at 4° C.;
Remove supernatant;
Add 300 μL ethanol 70% (−20° C.);
Centrifuge 12,500 rpm 15 min at 4° C.;
Remove gently the supernatant;
Let it dry;
Water resuspension (˜20 μL).

For medium scale preparation, plasmidic DNA was prepared from 100 mL of each culture, containing the same number of cells, using the Qiagen maxi prep kit (QIAGEN®), following the manufacturers' instructions or alternatively by dividing the volume for each buffer by 4. In all cases, the preparations of DNA were resuspended in 500 μL of water.

Analysis of the recovered nucleic acids were performed by electrophoresis.

### 2—Results

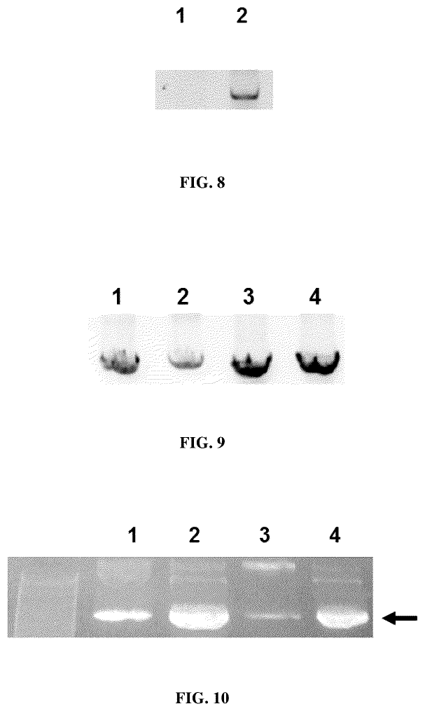

To determine whether the decreased resistance to osmotic shock correlated with the amount of extract chromosomal DNA recovered using standard extraction protocol, the recovery of plasmidic DNA in small scale preparations was assayed in the MG1655 E. coli wild type strain or a strain bearing the combination of mutations lppΔK58 ompAR256E. The first protocol used, using non-commercial conditions (see above), did not allow the recovery of plasmidic DNA from the control E. coli stain. In contrast, when using E. coli with the mutations lppΔK58 ompAR256E, plasmidic DNA was detected by electrophoresis (FIG. 8).

Medium scale preparation of plasmidic DNA from the E. coli MG1655 strain and the E. coli strain lppΔK58 ompAR256E were then tested using either the recommended volumes of P1, P2, and N3 buffers or dividing this volume by 4 to determine whether plasmidic DNA could be recovered using smaller amount of lysis buffer. The preparations were quantified and their content analyzed by electrophoresis (FIG. 9). In the condition using the recommended volume of P1, P2 and N3 buffers, the amount of plasmidic DNA recovered was larger when using the E. coli strain lppΔK58 ompAR256E (10⁵ ng/μL) than the amount recovered from the control strain (70 ng/μL). Similarly, in the condition using ¼ of the volume of P1, P2 and N3 buffers, the amount of plasmidic DNA recovered was larger when using the E. coli strain lppΔK58 ompAR256E (110 ng/μL) than the amount recovered from the same number of control cells (30 ng/μL). When using the recommended volume of buffer, the increase in the amount of plasmidic DNA recovered with the E. coli strain lppΔK58 ompAR256E was 1.5-fold when compared with the control MG1655 E. coli strain. When using ¼ of the recommended volume of P1, P2 and N3 buffers, the increase in the amount of plasmidic DNA recovered with the E. coli strain lppΔK58 ompAR256E was 3,66-fold when compared with the control E. coli MG1655 strain. On note, the lysate when preparing plasmid from E. coli strain lppΔK58 ompAR256E was clearer and less viscous, suggesting that upon lysis, the bacterial cells released less genomic DNA.

### 3—Conclusion

The use of E. coli MG1655 strain lppΔK58 ompAR256E allows increasing the amount of plasmidic DNA recovered in both small scale preparation and medium scale preparation when compared to the control wild type MG1655 E. coli strain. This increase was higher when smaller volumes of lysis buffers were used. The latter observation indicates that an E. coli strain of genotype lppΔK58 ompAR256E would be particularly advantageous in larger scale extrachromosomal DNA preparations as it would allow to decrease the amount of required lysis buffer.

### Example 4. The Use of E. coli Strain DH10B and DH5α of Genotype LppΔK58 ompAR256E Increases the Recovery of Extra-Genomic DNA

### 1—Materials and Methods

### 1.1—Bacterial Culture and Preparation of Competent Cells

Exponential phase culture grown in LB (1% tryptone, 0.5% yeast extract, 1% NaCl) at 37° C. under agitation were washed several times in cold Water supplemented with Sorbitol and kept on ice before electroporation. Whenever necessary, antibiotics were used at 25 μg/mL (kanamycin; Kan), 15 μg/mL (chloramphenicol; Cm) or 100 μg/mL (ampicillin; Amp).

### 1.2—Recovery of Plasmidic DNA in Large Scale Preparations

The plasmid pGWIZ gWiz™ Vectors was transformed into either a control of E. coli of genotype lppΔK58 ompAR256E of strain DH10B or DH5α using the method described in Macherey-Nagel NucleoBond® Xtra Midi.

### 2—Results

We determined whether the increased amount of extra-genomic DNA recovered from preparation from E. coli MG1655 strain lppΔK58 ompAR256E was also observed in the E. coli strains DH10B and DH5α. Preparation of plasmidic DNA from the control E. coli DH10B and DH5α strains and the E. coli DH10B and DH5α strains bearing the mutations lppΔK58 and ompAR256E were measured dividing by 8 the recommended volume of lysis buffer. The preparations were analyzed by electrophoresis and further quantified (FIG. 10). The amount of plasmidic DNA recovered was larger when using the E. coli DH10B strain lppΔK58 ompAR256E (521 ng/μL) than the amount recovered from the control strain (225 ng/μL). Similarly, the amount of plasmidic DNA recovered was larger when using the E. coli DHα strain lppΔK58 ompAR256E (643 ng/μL) than the amount recovered from the control strain (191 ng/μL). The increase in the amount of plasmidic DNA recovered with the E. coli strain DH10B lppΔK58 ompAR256E was of 2.3-fold when compared with the control E. coli DH10B strain. The increase in the amount of plasmidic DNA recovered with the E. coli strain DH5α lppΔK58 ompAR256E was of 3.36-fold when compared with the control E. coli DH5a strain.

### 3—Conclusion

The use of DH10B or DH5α E. coli strains lppΔK58 ompAR256E increases the amount of plasmidic DNA recovered when compared to control E. coli DH10B and DH5α strains when the recommended volume of lysis buffer was divided by 8. In addition, the amount of plasmidic DNA recovered from mutated DH5α E. coli strains was superior to the amount recovered from mutated DH10B E. coli, in similar conditions.

### Example 5. Effect of Mutation(s) Affecting the Bacterial Cell Wall on Protein Release after Osmotic Shock

### 1—Materials and Methods

### 1.1—Bacterial Culture

E. coli strains were grown in LB (1% tryptone, 0.5% yeast extract, 1% NaCl) at 37° C. under agitation.

### 1.2—Quantification of the Release of Protein in the Culture Medium after Osmotic Shock

Exponential or stationary culture phase E. coli strains were pelleted and resuspended in twice their initial volume with pure water for 5 minutes. 15 μL of the resuspension were then analyzed by SDS-PAGE electrophoresis. Proteins were further quantified by Coomassie blue staining.

### 2—Results

The amount of total proteins released after osmotic shock, by the bacterial cell with the mutations Δlpp or ΔompA or Δlpp ompAΔc was higher than the amount released by the same number of bacterial cells of control E. coli strain MG1655. The amount of bacterial cell released by the bacterial cell having the genotype Δlpp ΔompA or Δlpp ompAΔc was also higher than the amount released by the same number of bacterial cells of genotype Δlpp.

### 3—Conclusion

The use of E. coli MG1655 strains of genotype Δlpp ΔompA or Δlpp ompAΔc increases the amount of protein released by bacterial cells after an osmotic shock when compared to control E. coli MG1655 strains or E. coli MG1655 strain of genotype Δlpp.

### Example 6. The Use of E. coli Strain DH10B of Genotype ompAR256E, of Zenotype LppΔK58 and of Genotype ompAR256E LppΔK58, all Increase the Recovery of Extra-Zenomic DNA

### 1—Materials and methods

Bacterial strains and DNA recovery was performed as described in example 3.

### 2—Results

As seen in Table 4, the double mutant ompAR256E lppΔK58 results a significant increase of DNA recovery, as compared to the wild type reference strain (DH10B).

Surprisingly, single mutation, ompAR256E and single mutation lppΔK58, also resulted in an increased amount of DNA recovery as compared to the wild type strain (see Table 4).

TABLE 4
DNA recovery yield in single mutants ompAR256E and lppΔK58

| | Final | Weight | EB | Recovered |
|---|---|---|---|---|
| Strain | OD | (g) | (ml) | DNA (ng/μl) |
| DH10B | 2.17±0.50 | 0.48±0.02 | 0.1 | 470±117 |
| DH10B ompAR256E | 1.43±0.40 | 0.41±0.06 | 0.1 | 781±66 |
| lppΔK58 | | | | |
| DH10B ompAR256E | 1.83±0.58 | 0.43±0.02 | 0.1 | 670±114 |
| DH10B lppΔK58 | 2.23±0.59 | 0.45±0.04 | 0.1 | 600±126 |

Consequently, E. coli strains with ompAR256E mutation and/or lppΔK58 mutation may be useful to obtained increased amount of extra genomic nucleic acids.

### Example 7. The Use of E. coli Strain MG1655 of Genotype LppΔK58 ompAΔCt Increases the Recovery of Extra-Genomic DNA

### 1—Materials and Methods

Bacterial strains and DNA recovery was performed as described in example 3.

### 2—Results

As seen in Table 5 below, E. coli strains with ompAΔCt lppΔK58 double mutation results in a significant increase of the amount of recovered nucleic acid, as compared to the wild type strain.

TABLE 5
DNA recovery yield in double mutant ompAΔCt lppΔK58

| | Final | Weight | EB | Recovered |
|---|---|---|---|---|
| Strain | OD | (g) | (ml) | DNA (ng/μl) |
| MD243 (XL1 Blue) | 3.53±1.40 | 0.54±0.02 | 0.1 | 115±12 |
| MD1236 ompAΔCt | 3.47±1.07 | 0.53±0.06 | 0.1 | 155±35 |
| lppΔK58 | | | | |

Consequently, E. coli strains with ompAΔCt lppΔK58 double mutation may be useful to obtained increased amount of extra genomic nucleic acids.

1-33. (canceled)

34. A genetically modified Escherichia coli bacterium comprising at least two mutated genes encoding proteins involved in the envelope integrity, said bacterium having an altered envelop integrity and being oversensitive to bacterial lysis as compared to a bacterium with unaltered envelop integrity, wherein at least one mutated gene is ompA, and/or a homologue thereof, and at least one mutated gene is a gene involved in Lpp functionality, with the proviso that the bacterium does not comprise simultaneously a complete deletion of the ompA gene and a complete deletion of the lpp gene.

35. The bacterium according to claim 34, wherein the at least one gene involved in Lpp functionality is selected in the group comprising or consisting of lpp, ybiS, ycfS and erfK genes, and/or homologues thereof, and any combinations thereof.

36. The bacterium according to claim 34, wherein said at least two mutated genes comprise one of the following combinations:
ompA and lpp, and/or a homologue thereof;
ompA and ybiS, and/or ycfS and/or erfK, and/or a homologue thereof;
ompA, lpp, ybiS and erfK, and/or a homologue thereof;
ompA, lpp, ycfS and erfK, and/or a homologue thereof; or,
ompA, lpp, ybiS and ycfS, and/or a homologue thereof.

37. The bacterium according to claim 34, wherein the mutated ompA gene comprises a substitution of the codon encoding arginine (R) at position 256 with a codon encoding a neutrally or negatively charged amino acid; and/or a substitution of the codon encoding aspartic acid (D) at position 241 with a codon encoding a neutrally or positively charged amino acid; and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

38. The bacterium according to claim 34, wherein the mutation in the lpp gene is selected in the group comprising, or consisting of, a deletion of the codon encoding lysine (K) at position 58; a substitution of the codon encoding arginine (R) at position 57 with a codon encoding another amino acid; a substitution of the codon encoding lysine (K) at position 58 with a codon encoding an arginine (R); a complete deletion of the lpp gene; and combinations thereof, wherein said positions are defined with respect to the amino acid sequence SEQ ID NO: 6.

39. The bacterium according to claim 34, wherein the mutated ybiS gene, ycfS gene and/or erfK gene, and/or a homologue thereof, consist in a deletion of said ybiS, ycfS and/or erfK genes, and/or a homologue thereof, respectively.

40. The bacterium according to claim 34, wherein said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; and a mutation in the lpp gene consisting of the deletion of the codon encoding lysine (K) at position 58, wherein said position is defined with respect to the amino acid sequence SEQ ID NO: 6.

41. The bacterium according to claim 34, wherein said bacterium has a deletion of the C-terminal part of the OmpA protein starting at or before the codon encoding aspartic acid (D) at position 241 or arginine (R) at position 256, wherein said position is defined with respect to the amino acid sequence SEQ ID NO: 3; and a mutation in the lpp gene consisting of the deletion of the codon encoding lysine (K) at position 58, wherein said position is defined with respect to the amino acid sequence SEQ ID NO: 6.

42. The bacterium according to claim 34, wherein said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; a complete deletion of the ybiS gene; a complete deletion of the ycfS gene; and complete deletion of the erfK gene.

43. The bacterium according to claim 34, wherein said bacterium has a mutation in the ompA gene consisting of the substitution of the codon encoding arginine (R) at position 256 with a codon encoding a glutamic acid (E), wherein said position is defined with respect to the amino acid sequence SEQ ID NO: 3; a mutation in the lpp gene consisting of the substitution of the codon encoding arginine (R) at position 57 with a codon encoding a leucine (L), wherein said position is defined with respect to the amino acid sequence SEQ ID NO: 6; a deletion of each of the ybiS gene; and a complete deletion of the ycfS gene.

44. The bacterium according to claim 34, wherein said bacterium has a mutation in the ompA gene consisting of a substitution of the codon encoding aspartic acid (D) at position 241 with a codon encoding asparagine (N), wherein said position is defined with respect to the amino acid sequence SEQ ID NO: 3; and a complete deletion of the lpp gene.

45. The bacterium according to claim 34, wherein said bacterium further comprises at least one extra-genomic nucleic acid molecule.

46. A method for the production and the purification of at least one extra-genomic nucleic acid molecule or at least one polypeptide comprising the steps of:
a) culturing genetically modified E. coli bacteria comprising at least one mutated gene encoding a protein involved in the envelope integrity, said bacteria having an altered envelop integrity and being oversensitive to bacterial lysis as compared to a bacterium with unaltered envelop integrity, wherein the at least one mutated gene is ompA, and/or a homologue thereof, or a gene involved in Lpp functionality, said bacteria comprising at least one extra-genomic nucleic acid molecule or comprising a nucleic acid molecule encoding at least one polypeptide, so as to amplify the at least extra-genomic nucleic acid molecule or the at least one polypeptide;
b) lysing the bacteria obtained at step a) so as to obtain a lysis mixture; and,
c) purifying said amplified extra-genomic nucleic acid molecule or said at least one polypeptide from the lysis mixture obtained at step b).

47. The method according to claim 46, wherein the at least one extra-genomic nucleic acid molecule is selected in the group comprising or consisting of a plasmid, a cosmid and a bacterial artificial chromosome (BAC).

48. The method according to claim 46, wherein the at least one polypeptide is one cytoplasmic polypeptide.

49. The method according to claim 46, wherein the at least mutated ompA gene consists of a substitution of the codon encoding arginine (R) at position 256 with a codon encoding a neutrally or negatively charged amino acid; and/or a substitution of the codon encoding aspartic acid (D) at position 241 with a codon encoding a neutrally or positively charged amino acid; and/or a deletion of the C-terminal part of the OmpA protein starting at or before the codon encoding aspartic acid (D) at position 241 or arginine (R) at position 256; or a complete deletion of the ompA gene; wherein said positions are defined with respect to the amino acid sequence SEQ ID NO: 3.

50. The method according to claim 46, wherein the at least mutated gene involved in Lpp functionality consists of a mutation in the lpp gene consisting of the deletion of the codon encoding lysine (K) at position 58, wherein said position being defined with respect to the amino acid sequence SEQ ID NO: 6, or the complete deletion of ybiS gene, and/or the complete deletion of the ycfS gene and/or the complete deletion of the erfK gene.

51. The method according to claim 46, wherein the bacterium comprises at least two mutated genes encoding proteins involved in the envelope integrity, and wherein at least one mutated gene is ompA, and/or a homologue thereof, and at least one mutated gene is a gene involved in Lpp functionality.

52. The method according to claim 51, wherein the bacterium does not comprise simultaneously a complete deletion of the ompA gene and a complete deletion of the lpp gene.

53. A kit comprising (i) a genetically modified E. coli bacterium comprising at least one mutated gene encoding a protein involved in the envelope integrity, said bacterium having an altered envelop integrity and being oversensitive to bacterial lysis as compared to a bacterium with unaltered envelop integrity, wherein the at least one mutated gene is ompA, and/or a homologue thereof, or a gene involved in Lpp functionality; and (ii) means to transform said bacterium with an extra-genomic nucleic acid molecule.
